(19) United States
(12) Patent Application Publication
(10) Pub. No.: US 2016/0157739 A1
(43) Pub. Date: Jun. 9, 2016

(54) DETERMINING RETURN OF SPONTANEOUS CIRCULATION DURING CPR

(71) Applicant: KONINKLIJKE PHILIPS N.V., Eindhoven (NL)

(72) Inventors: WOUTER HERMAN PEETERS, WAALRE (NL); RALPH WILHELM CHRISTIANUS GEMMA WIJSHOFF, MUNSTERGELEEN (NL); ANTOINE MICHAEL TIMOTHY MARIA VAN ASTEN, AMSTERDAM (NL); RICK BEZEMER, AMSTERDAM (NL); RONALDUS MARIA AARTS, GELDROP (NL); PIERRE HERMANUS WOERLEE, VALKENSWAARD (NL)

(21) Appl. No.: 14/905,914

(22) PCT Filed: Feb. 4, 2015
(86) PCT No.: PCT/EP2015/052227

§ 371 Date: Jan. 18, 2016

(30) Foreign Application Priority Data
Feb. 11, 2014 (EP) 14154681.2
Jul. 17, 2014 (EP) 14177397.8

(51) Int. Cl. A61B 5/024; A61H 31/00; A61N 1/39; A61B 5/00

(52) U.S. Cl. CPC A61B 5/02416; A61B 5/7275; A61H 31/006; A61B 5/4836; A61N 1/3937

(57) ABSTRACT

There is provided a device (100) and method for determining a total probability (Ptot) of Return of Spontaneous Circulation (ROSC) during an associated CPR procedure, which is being performed on an associated patient, comprising an input for receiving a set of photoplethysmograpy data (328, 330) having been obtained from the associated patient during the CPR procedure, and a processor (112) being arranged for carrying out one or more processes according to one or more predetermined algorithms (321, 322, 323, 324) so as to calculate the total probability (Ptot) of ROSC based on the one or more parameters, wherein the one or more processes are each, and/or in combination, being arranged for overcoming challenges derived from the CPR process, such as arbitrary signals not related to return of spontaneous circulation. In embodiments, the device and method relies on a plurality of processes in determining the total probability of ROSC.

### FIELD OF THE INVENTION

The present invention relates to the field of determining return of spontaneous circulation, in particular the invention relates to a device, method and computer program for determining return of spontaneous circulation during cardiopulmonary resuscitation.

### BACKGROUND OF THE INVENTION

Cardiopulmonary resuscitation for cardiac-arrest patients is an emergency procedure with a very low survival rate (5-10%). It is commonly accepted that the quality of the chest compressions is of crucial importance for successful defibrillation and outcome.

The reference US 2012/0035485A1 describes that the presence of a cardiac pulse in a patient is determined by evaluating physiological signals in the patient. In one embodiment, a medical device evaluates optical characteristics of light transmitted into a patient to ascertain physiological signals, such as pulsatile changes in general blood volume proximate a light detector module. Using these features, the medical device determines whether a cardiac pulse is present in the patient. The medical device may also be configured to report whether the patient is in a VF, VT, asystole, or PEA condition, in addition to being in a pulseless condition, and prompt different therapies, such as chest compressions, rescue breathing, defibrillation, and PEA-specific electrotherapy, depending on the analysis of the physiological signals. Auto-capture of a cardiac pulse using pacing stimuli is further provided. Reference W.C.G.R. et Al: “Detection of a spontaneous pulse in photoplethysmograms during automated cardiopulmonary resuscitation in a porcine model”, RESUSCITATION, vol. 84, 2013, pages 1625-32, XP55125349 discloses an investigation of the potential of photoplethysmograms (PPG) to detect the presence and rate of a spontaneous cardiac pulse during CPR, by retrospectively analyzing PPG and arterial blood pressure signals simultaneously recorded in pigs undergoing automated CPR. Reference GUNDERSEN K et Al: “Chest compression quality variables influencing the temporal development of ROSC-predictors calculated from the ECG during VF”, RESUSCITATION, ELSEVIER, IE, vol. 80, no. 2, 1 Feb. 2009 (2009 Feb. 1), pages 177-182, XP025771817, ISSN:0300-9572, DOI: 10.1016/J.RESUSCITATION.2008.09.011 [retrieved on 2008 Dec. 6] discloses the concept of formulating a model for the influence of CPR and compression quality variables, on the temporal development of one “return of spontaneous circulation” (ROSC) predictor: median slope. This is a feature that can be extracted from an electrocardiogram during ventricular fibrillation and ventricular tachycardia and can, to a certain extent predict ROSC upon fibrillation.

However, it may be seen as an objective to minimize disadvantages associated with interruptions of the chest compression sequence being performed on an associated patient.

Hence, an improved device, method and computer program enabling minimizing disadvantages associated with interruptions of the chest compression sequence would be advantageous.

### SUMMARY OF THE INVENTION

It would be advantageous to provide an improved device, method and computer program enabling minimizing disadvantages associated with interruptions of the chest compression sequence. It is a further object of the present invention to provide an alternative to the prior art.

In a first aspect, the invention provides a device for determining a total probability of Return of Spontaneous Circulation during an associated CPR procedure which is being performed on an associated patient, the device comprising:

an input for receiving a set of photoplethysmograpy data having been obtained from the associated patient during the CPR procedure,

a processor being arranged for
accessing the photopletysmography data,
carrying out one or more processes according to one or more predetermined algorithms, such as one or more automatable algorithms, such as one or more processes which do not require user input, based on the photoplethysmography data, so as to calculate one or more parameters (PA, PB, PC, PD) indicative of a probability of Return of Spontaneous Circulation corresponding to an outcome of each process within the one or more processes, and
calculating the total probability (Ptot) of Return of Spontaneous Circulation based on the one or more parameters (PA, PB, PC, PD) indicative of a probability of Return of Spontaneous Circulation corresponding to an outcome of each process within the one or more processes,

an output arranged for providing a Return of Spontaneous Circulation probability signal based on the total probability (Ptot) of Return of Spontaneous Circulation.

The present invention may be beneficial for mitigating the problems with pulse check pauses by providing a method and device that may quickly and/or accurately and/or automatically determine a probability of Return of Spontaneous Circulation during an associated CPR procedure. For example, an advantage of the present invention may be that it may enable preventing futile detrimental pulse checks, thereby potentially mitigating the effects of (unnecessary) pulse check pauses. It is noted, that previous references may focus on improving the time and quality of pulse checks (as opposed to avoiding those pulse checks which are unnecessary). More particularly, accessing the photopletysmography data having been obtained from the associated patient during the CPR procedure, carrying out one or more processes according to one or more predetermined algorithms and calculating the total probability of Return of Spontaneous Circulation, may enable a user to gain insight into whether or not it makes sense to interrupt the CPR procedure and carry out a pulse check. Embodiments of the invention may enable prompting a caregiver to provide appropriate therapy in an emergency situation.

It is noted that one of the quality aspects of the chest compressions is minimization of interruptions of the chest compression sequence. A commonly-accepted type of interruption is the “pulse-check pause”, such as a pause in which the caregiver manually touches the neck of the patient to determine absence or presence of pulsations in the carotid artery. To minimize the duration of this type of pauses, clinical guidelines state that a pulse-check pause should take no longer than 10 seconds.

In clinical practice, manual pulse checks often take much longer than 10 seconds and are known to be unreliable even if performed by expert clinicians. There is a need for a fast, automated, and accurate method to do a pulse check, as to reduce the duration of the pauses and to reduce the amount of false pulse determinations. Recording of the electrocardiogram (ECG) alone does not provide the information as the heart may be electrically active but may not produce cardiac output.

In the scientific literature, people have been investigating various physiological signals that could be used to detect presence or absence of pulse. Monitoring of end-tidal CO₂, invasive blood pressure, or central venous oxygen saturation, allows for an objective assessment of pulse, but requires a secured airway or placement of catheters. Transthoracic impedance (TTI) measurements, and near-infrared spectroscopy (NIRS) are non-invasive, but TTI is strongly influenced by chest compressions and NIRS responds slowly upon ROSC.

Use of photoplethysmography data has previously been described as not reliable. References on photoplethysmography data for pulse detection exist, but do not disclose one or more algorithms, such as one or more automatable algorithms, such as one or more algorithms which do not require user input (such as visual input) which enable calculating a probability of Return of Spontaneous Circulation based on the photoplethysmography data having been obtained from the associated patient during the CPR procedure.

During chest compressions physiological signals cause methods of the prior art to malfunction, the reason is that chest compressions generate significant signal artefacts (e.g., due to the compressions), which must be distinguished from true cardiac pulses. It may be understood that predetermined algorithms, such as predetermined algorithms enabling automatable processes, are beneficial for distinguishing compression-induced features in the signals from the cardiac-induced features. Therefore, determining the (total) probability on Return of Spontaneous Circulation during chest compressions, such as during a CPR procedure, is advantageously carried out using such predetermined algorithms. Preventing futile pulse checks, rather than shortening pulse checks, thus requires advanced algorithms that are reliable during the chest compression sequence. It may be noted, that the ability to enable providing, such as enable automatically providing, a ROSC probability signal based on data obtained during a CPR procedure, may be seen as an advantage over prior art references.

In embodiments of the present invention there is presented a device which is capable of presenting advice pro- or con stopping the compression sequence for a pulse check during chest compressions, such as during a CPR procedure, and optionally also during pauses in the compression sequence.

References featuring the present inventors, such as the reference Wijshoff, R. W. C. G. R. et al. Detection of a spontaneous pulse in photoplethysmograms during automated cardiopulmonary resuscitation in a porcine model. Resuscitation 84, 1625-32 (2013), which is hereby incorporated by reference in entirety, and the reference, Wijshoff, R., Van der Sar, T., Aarts, R., Woerlee, P. & Noordergraaf, G. Potential of photoplethysmography to guide pulse checks during cardiopulmonary resuscitation: Observations in an animal study. Resuscitation 84, S1 (2013), which is hereby incorporated by reference in entirety, deals with photoplethysmography in relation to CPR. However, the present invention is advantageous at least in that it includes a processor arranged for carrying out one or more processes according to one or more predetermined algorithms and calculating the total probability of Return of Spontaneous Circulation, so as to enable the device to output the total probability of Return of Spontaneous Circulation, such as enables rendering user input, such as visual inspection unnecessary.

In the present application, compressions could be exchanged with decompressions, i.e., any occurrence of ‘compression’ could be exchanged with ‘compression and/or decompression’.

A patient can only have Return of Spontaneous Circulation (ROSC) when a perfusing rhythm has been re-established, i.e., when the heart contracts again at a stable rate, resulting in cardiac output. Therefore, by detecting the pulse rate, one may provide the clinician with information about the rate at which the heart contracts and pumps blood. If this rate is too low, e.g., when the rate is below 1 Hz, the clinician can decide that there is no ROSC yet and that delivering chest compressions should be continued. Furthermore, when the detected pulse rate varies too much over time, this may indicate that the heart is not yet pumping in a stable fashion. This information can also be of use to the clinician to help him decide how to continue the CPR process. When the heart is pumping again at a stable rate higher than, e.g., 1 Hz, he can decide to further examine whether there is ROSC, by doing additional measurements (e.g., blood pressure, or end-tidal CO₂). Presence of a stable pulse rate which is sufficiently high therefore is a prerequisite of ROSC: without such a rhythm, there will be no ROSC, and it will be of no use to do a further assessment of ROSC. On the other hand, presence of a stable, sufficiently high pulse rate in the PPG signal does not directly indicate that there is ROSC, because it does not provide the clinician with the information about the underlying blood pressure and/or level of perfusion. Additional measurements are required to determine this. Nonetheless, via embodiments of the present invention one can easily, and non-invasively obtain information about presence or absence of a stable, perfusing rhythm at a sufficiently high rate. Therefore, via the PPG-based pulse rate measurement, such as via embodiments of the present invention, the clinician can decide whether or not to stop chest compressions and do a further assessment of ROSC.

‘Return of Spontaneous Circulation’ (ROSC) is understood as is known in the art, and refers to Clinical significance of return of pulse.

By ‘during an associated CPR procedure’ is understood, that the photoplethysmography data has been obtained during a CPR procedure, i.e., the data have been recorded across a time period where one or more CPR compressions and/or decompressions have been carried out, such as the data comprising compression artefacts.

By an ‘associated patient’ is understood the patient which is not part of the claimed subject-matter.

By ‘an input for receiving a set of photoplethysmograpy data’ may be understood a data interface capable of communicating said data, such as an analogue or digital interface, such as a wireless connection, such as a wired connection, such as a USB connection.

By ‘photoplethysmography (PPG) data’ may be understood physiological data derived from light-based techniques (e.g., a pulse oximetry signal), such as light transmitted through the patient's tissue, such as tissue being and/or including skin, such as data obtained by illuminating the tissue and measuring changes in light absorption and/or reflection. PPG measurements can be carried out non-invasively at the tissue surface, where the light source and detector can be in contact with the tissue. PPG measurements can also be carried out at a distance from the tissue, where the light source and/or detector are not in contact with the tissue, such as in the case of camera-based measurements. The PPG data may be obtained at one or more wavelengths, such as 1, 2, 3, 4, 5, 6, 7, 8, 9, 10 or more wavelengths. In an embodiment, the incoming light is ambient light, such as sunlight. In an embodiment, PPG data may be obtained using a pulse oximeter which monitors the perfusion of blood, such as monitors the perfusion of blood to the dermis and subcutaneous tissue of the skin, and/or monitors the perfusion of blood through mucosal tissue. Apparatus and techniques for obtaining PPG data, such as pulse oximetry data, are well known in the art. Pulse oximetry is described in the reference US 2012/0035485 A1 which is hereby incorporated in entirety by reference.

One suitable system for obtaining PPG data includes a sensor with a red LED, a near-infrared LED, and a photodetector diode, where the sensor is configured to place the LEDs and photodetector diode directly on the skin of the patient, typically on a digit (finger or toe) or earlobe. Other places on the patient may also be suitable, including the forehead, the nose or other parts of the face, the wrist, the chest, the nasal septum, the alar wings, the ear canal, and/or the inside of the mouth, such as the cheek or the tongue. The LEDs emit light at different wavelengths, which light is diffused through the vascular bed of the patient's skin and received by the photodetector diode. The resulting PPG signal may then be analyzed for one or more features indicative of a cardiac pulse. Other simpler versions of a system for obtaining PPG data may be used, including a version with a single light source of one or more wavelengths. The absorption or reflectance of the light is modulated by the pulsatile arterial blood volume and detected using a photodetector device. In an embodiment, PPG data can be obtained from camera images, where ambient light and/or additional light sources are used to illuminate the tissue, such as skin.

It is noted, that in alternative embodiments of the invention, the PPG data may be replaced by other physiological data relating to cardiac pulse. Thus, in an alternative embodiment of the invention, PPG data may be replaced by ‘physiological data relating to the cardiac pulse’, for example (in parentheses are indicated processes within processes A-D described below, which the data type is particularly suitable for) ‘phonocardiogram data’ (AB), ‘electrocardiogram data’ (AB), ‘transthoracic impedance data’ (AB) and/or ‘infra-arterial blood pressure data’ (ABC). An advantage of PPG data may be that it is applicable for each and all of processes A, B, C and D described below.

By ‘having been obtained from the associated patient’ may be understood that the data may be obtained. It is also understood, that the claim does not comprise a step of interaction with the patient. It is in general noted that the invention is not about providing a diagnosis or about treating patients, but rather about a technical invention that solves a technical problem and that provides an output that may assist a physician in reaching a diagnosis or treating a patient.

By ‘one or more predetermined algorithms’ may be understood one or more automatable algorithms, such as one or more predetermined algorithms enabling automated processes, such as one or more algorithms or processes which do not require user input, based on the photoplethysmography data obtained during a CPR procedure. The algorithms may be understood to be predetermined in the sense that they can be implemented in a computer program product, but it also encompassed that they can be modified during use, e.g., that a weighting factor in a formula may be adjusted in dependence of input from and/or to a predetermined algorithm. The algorithms may be implemented in a computer program product. The algorithm may enable the device to function, even in the absence of user input. This may be seen as an advantage over prior art references which may necessitate user input, such as input based on visual assessment of data. An advantage of not needing user input, may be that the user need not spend time on, e.g., assessing a photoplethysmogram, such as during a CPR procedure.

By ‘one or more parameters indicative of a probability of Return of Spontaneous Circulation’ may be understood a number which is indicative of a probability of Return of Spontaneous Circulation.

By ‘an output arranged for providing a Return of Spontaneous Circulation probability signal’ may be understood a data interface capable of communicating said signal, such as an analogue or digital interface, such as a wireless connection, such as a wired connection, such as a USB connection. In an embodiment, the output may comprise audio-signals and/or visual signals.

By ‘calculating the total probability (Ptot) of Return of Spontaneous Circulation based on the one or more parameters (PA, PB, PC, PD) indicative of a probability of Return of Spontaneous Circulation corresponding to an outcome of each process within the one or more processes’ may be understood, that the processor receives the parameters and calculates the total probability (Ptot) of Return of Spontaneous Circulation based on said parameters. In the following, exemplary embodiments for calculating the total probability (Ptot) of Return of Spontaneous Circulation are presented in the exemplary context of the parameters corresponding to one or more of the processes A-D described below, wherein embodiments for calculating the parameters are also addressed.

The exemplary embodiments can be separated into at least four categories:

### (1) Simplest

a. methods for computing each of {PA,PB,PC,PD} without using CPR data and/or defibrillator data and/or memory unit
b. method for combining {PA,PB,PC,PD} without using CPR data and/or defibrillator data and/or memory unit.

### (2) Advanced Processes

a. methods for computing each of {PA,PB,PC,PD} using CPR data and/or defibrillator data and/or memory unit
b. method for combining {PA,PB,PC,PD} without using CPR data and/or defibrillator data and/or memory unit.

### (3) Advanced Combination

a. methods for computing each of {PA,PB,PC,PD} without using CPR data and/or defibrillator data and/or memory unit,
b. method for combining {PA,PB,PC,PD} using CPR data and/or defibrillator data and/or memory unit,

### (4) Advanced Processes and Combination

a. methods for computing each of {PA,PB,PC,PD} using CPR data and/or defibrillator data and/or memory unit
b. method for combining {PA,PB,PC,PD} using CPR data and/or defibrillator data and/or memory unit.

The embodiments are elaborated below:

### (1) Simplest

The method for combining {PA, PB, PC, PD} without using CPR data and/or defibrillator data is in the form of

Ptot=f(PA,PB,PC,PD)

where Ptot is the combined probability, {PA, PB, PC, PD} are the probabilities of ROSC resulting from the individual processes, and f(x) is a mathematical function. One embodiment of the function is for example

f(PA,PB,PC,PD)=[(PC>ThresholdC)+2*(PA>ThresholdA)+(PB>ThresholdB)+0.3*(PD>ThresholdD)]/4.3

where we used thresholding and logical operations. The outcome of the probability is in this case separated in a couple of discrete levels. Another embodiment of the function is

f(PA,PB,PC,PD)=[2*(PA>ThresholdA)+(1−(PA>ThresholdA))*(PB>ThresholdB)]/2

where we actually did not utilize processes C and D.

In yet another embodiment, the function generates an output on a continuous scale between 0 and 1:

f(PA,PB,PC,PD)=[1−exp(−PB/w_B)]*[1−exp(−PC/w_C)]

where we only use process B and C, scalar weights w_B and w_C.

### (2) Advanced Processes

We speak of “advanced processes” if it makes use of memory units, i.e., adaptive parameters that are stored in memory that modify the computation of {PA, PB, PC, PD} or if it uses defibrillation timing or CPR data. For example, in one of our embodiments Process C keeps track (within a memory unit) of a flag that is only changed if the PPG-baseline change rate crosses certain thresholds. In this embodiment we combine the processes as follows

f(PA,PB,PC,PD)=(PC>ThresholdC)*[(PA>ThresholdA)+(PB>ThresholdB)+1]

Using a memory unit is also known as using a ‘finite state machine’, in a sense that the memory unit remembers the state in which the machine resides.

We also speak of ADVANCED PROCESSES if one uses defibrillation timing data (such as make use of the knowledge of the point in time in which a defibrillation event occurred, such as coordinate t-t_defib) or CPR data for the computation of PA, PB, PC and PD. For example, one can store the PPG baseline level in a memory unit at the moment of defibrillation, and one can weight the importance of baseline drifts with how recent the last defibrillation attempt was. Also, CPR data may be especially useful in all processes to distinguish between periods where compressions are present and periods where compressions are absent. Precise compression timing is particularly useful in process A where it is required to know the compression frequency.

### (3) Advanced Combinations

The combination function now becomes a function of three more variables Ptot=f(PA, PB, PC, PD, t-t_defib, Compression Depth, Compression Force)

incorporating a time (t) coordinate relative to the last defibrillation event (t_defib), the compression depth, and the compression force. Other variables that can be used, include compression velocity and/or compression acceleration.

### (4) Advanced Processes and Advanced Combination

A combination of the advanced methods as described in (2) and (3).

In an embodiment, there is presented a device wherein the one or more processes comprise process A, wherein process A is a process comprising:

i. obtaining a spectrally resolved representation of the photoplethysmograpy data,
ii. identifying peaks in the spectrally resolved representation,
iii. identifying a chest compression frequency,
iv. scoring each peak, where a higher score is given where a higher number (such as for a higher number) of remaining peaks which correspond to a harmonic of the peak or correspond to a sum or difference frequency between

1. the peak or harmonics of the peak and

2. a chest compression frequency or harmonics of the chest compression frequency,

v. calculating a pulse rate within the data based on the peak with the highest score,
vi determining a process A parameter (PA) indicative of a probability of Return of Spontaneous Circulation based on said pulse rate, such as said pulse rate and the variability of the pulse rate, and optionally the amplitude of the peak with the highest score, such as the peak with the highest score being a peak corresponding to said pulse rate.

Chest compression frequencies may be known in embodiments, such as in case of automated CPR and/or when the device is arranged for receiving CPR data, and/or independently measured using, e.g., an accelerometer, a compression force measurement or means for providing transthoracic impedance data. An advantage of process A may be that it enables overcoming the challenges provided by

indistinguishability of compressions and heart rate in time representation, and

assessing clinical significance of strength of pulse.

In a further embodiment the scoring of each peak depends furthermore on

the amplitude of the peak, such as where a higher score is given for a higher amplitude, and/or

the amplitude of the remaining peaks, such as where a higher score is given for a higher amplitude, which correspond to a harmonic of the peak or correspond to a sum or difference frequency between

1. the peak or harmonics of the peak and

2. a chest compression frequency or harmonics of the chest compression frequency.

In a specific embodiment, the following signal model for the PPG signal during ongoing chest compressions is used:

PPG(t)=[sum{k=0}̂K A_k cos(2pi k f_prt+phi_k)]*[sum_{m=0}̂M B_m cos(2pi m f_cmp t+theta_m)],

in which the first series between square brackets describes the harmonic series of K pulse components at f_pr [Hz] and integer multiples thereof, with amplitude and phase terms A_k [Volt] and phi_k [rad], respectively, and in which the second series between square brackets describes the harmonic series of M compression components at f_cmp [Hz] and integer multiples thereof, with amplitude and phase terms B_m [Volt] and theta_m [rad], respectively. Here, t [s] represents time. Therefore, the following frequency components will be encountered in the PPG signal during ongoing chest compressions:

K pulse rate components: k f_pr, k=1, . . . , K

M compression rate components: m f_cmp, m=1, . . . , M

2*K*M interaction components: |k f_pr+−m f_cmp|, k=1, . . . , K, m=1, . . . , M

Based on this model, the peak with the highest score is the fundamental frequency of the pulse, because:

the compression rate and harmonics thereof are known frequencies, and can therefore be ignored in the analysis or removed from the signal prior to analysis,

for the remaining components, the highest number of harmonics will be found for the pulse rate fundamental, as it is the first component of the series,

if the pulse rate fundamental does not have any harmonics, it still can be recognized as the component right in the middle between the strongest interaction terms, e.g., between f_pr+f_cmp and |f_pr−f_cmp|. Here, the amplitude of the spectral components can be relevant, in order to be able to recognize the strongest interaction terms, which is why scoring may optionally be weighthed by peak amplitude.

In an embodiment, there is presented a device wherein the one or more processes comprise a process B, wherein process B is a process comprising:

i. obtaining a spectrally resolved representation of the photoplethysmograpy data for determining a measure of order, such as spectral entropy, of the photoplethysmography data, and
ii. calculating a process B parameter (PB) indicative of a probability of Return of Spontaneous Circulation based on said measure of order.

In an embodiment, said measure of order, is given by entropy, such as ‘Spectral entropy’. The spectral entropy is one way to quantify the structuredness of the spectrum mathematically. A specific embodiment uses the Shannon spectral entropy between 0 and 200 per minute. Other embodiments use similar but slightly different measures like for example Wiener Entropy/spectral flatness. An advantage of process B may be that it enables overcoming the challenges provided by

assessing clinical significance of strength of pulse, and

irregular beating of the heart in start-up phase

It may be noted that it may be seen as an advantage of process B, e.g., vs., process A, that process B is beneficial for overcoming challenges derived from irregular beating of the heart in start-up phase.

The present inventors have discovered that the heart beats very irregularly in the start-up phase just after de-fibrillation (irregular beating corresponds to very high entropy). Irregular beating in the start-up phase, was discovered to originate from the fact that not every R-peak in the electrical activity of the heart (ECG) results in an effective pulse in the blood stream. An advantage of process B and said measure of order, such as an entropy measure, in the context of PPG signals may be that is particularly effective for PPG signals, such as better than for other signals, such as ECG signals. A quickly rising spectral entropy, just after defibrillation indicates that the heart is starting up. It may take another minute or so before ROSC is fully reached. Nonetheless, it is important to distinguish a starting heart from a non-starting heart, as it is detrimental to use a vasopressor agent in the start-up phase of the heart, whereas a completely inactive heart may be treated with vasopressor agents.

In an embodiment, there is presented a device wherein the one or more processes comprise a process C, wherein process C is a process comprising:

i. determining a low-frequency value, such as a DC value, of the photoplethysmography data, and
ii. calculating a process C parameter (PC) indicative of a probability (PC) of Return of Spontaneous Circulation based on said low-frequency value.

Return of spontaneous circulation (ROSC) may correspond to an increase in central blood pressure. The low-frequency value, such as DC value, such as ‘baseline’, of the PPG signal may respond to changes in local blood pressure. The present embodiment, however, is based on the highly surprising insight, that the low-frequency value, such as DC value, such as ‘baseline’, of the PPG signal furthermore responds clearly to return of spontaneous circulation (ROSC). An advantage of process C may be that it enables overcoming the challenges provided by

assessing clinical significance of strength of pulse,

irregular beating of the heart in start-up phase, and

coinciding frequencies of compressions and heart rate

It may be noted that it may be seen as an advantage of process C, e.g., vs., process A, that process C is beneficial for overcoming challenges derived from irregular beating of the heart in start-up phase and for overcoming challenges derived from coinciding frequencies of compressions and heart rate.

In an embodiment, there is presented a device wherein the one or more processes comprise a process D, wherein the input is enabling receipt of the set of photoplethysmograpy data, where the set of photoplethysmography data is a set of photoplethysmography data obtained at different wavelengths, and wherein process D is a process comprising:

i. determining a level of correlation between the set of photoplethysmography data obtained at different wavelengths, and
ii. calculating a process D parameter (PD) indicative of a probability (PD) of Return of Spontaneous Circulation based on said level of correlation.

The present inventors have realized that the correlation may be used to assess the perfusion of the skin, and to assess the venous oxygen saturation. During cardiac arrest, the perfusion of the superficial layers of the skin may be poor. Upon ROSC, perfusion of the skin improves again, as observed during animal experiments (such as experiments with pigs): upon ROSC, the color of the skin (of the belly) of the pigs temporarily becomes more red. Furthermore, during cardiac arrest the venous oxygen saturation is low due to the reduced cardiac output, causing the venous blood to have a dark red color. Consequently, the absorption of the red light strongly increases, decreasing its penetration depth. Therefore, during cardiac arrest, the red light penetrates only the superficial, poor perfused layers of the skin, and that the infrared light also penetrates the deeper layers of the skin which are better perfused than the superficial layers. Therefore, as red and infrared light probe different tissue volumes which are presumably differently perfused during cardiac arrest, correlation between the signals is poor. After ROSC, the perfusion of the skin improves and the venous oxygen saturation increases again, causing the red and infrared light to probe comparable tissue volumes again, which improves the correlation between both PPG signals. An advantage of process D may be that it enables overcoming the challenges provided by

indistinguishable compressions and heart in time representation,

irregular beating of the heart in start-up phase, and

coinciding frequencies of compressions and heart rate.

It may be noted that it may be seen as an advantage of process D, e.g., vs., process A, that process D is beneficial for overcoming challenges derived from irregular beating of the heart in start-up phase.

In an embodiment, there is presented a device wherein the one or more processes comprise a plurality of processes, such as at least 2 processes, such as 2, 3, 4, 5, 6, 7, 8, 9, 10 processes, such as more than 10 processes. An advantage of a plurality of processes may be that a more reliable calculation of the total probability (Ptot) of Return of Spontaneous Circulation is provided, since more processes go into the calculation. An advantage of a plurality of processes may be that a confidence can be assigned to the total probability (Ptot) of Return of Spontaneous Circulation, depending on the differences in outcome of the individual processes. An advantage of a plurality of processes may be that the processes may supplement each other, such as some processes may meet certain challenges better than other processes, and vice versa.

In a further embodiment, there is presented a device wherein the one or more processes comprise at least one, such as 1, of the processes within processes A-D. In a further embodiment, there is presented a device wherein the one or more processes comprise 2 or 3 or 4 of the processes within processes A-D, such as 2, such as at least 3, such as 3, such as at least 4, such as 4 of the processes within processes A-D. In the following, the processes are referred to by their capital letter, such as process A, being ‘A’ and process A and process B being ‘AB’, etc. In an embodiment the one or more processes comprise 2 of the processes within processes A-D, such as AB, AC, AD, BC, BD, CD. In an embodiment the one or more processes comprise 3 of the processes within processes A-D, such as ABC, ABD, ACD, BCD. In an embodiment the one or more processes comprise 4 of the processes within processes A-D, such as ABCD.

In an embodiment, there is presented a device wherein the processor is furthermore arranged for:

calculating a risk parameter indicative of a risk that administration of a vasopressor agent would have negative effects, the risk parameter being based on the one or more parameters (PA, PB, PC, PD) indicative of a probability of Return of Spontaneous Circulation corresponding to an outcome of each process within the one or more processes, and wherein the output is furthermore arranged for

providing a vasopressor agent signal based on the risk parameter.

It may be difficult to decide whether or not to administer a vasopressor agent, e.g. epinephrine, to the patient. Vasopressor agents increase the probability of successful resuscitation if pulse is completely absent. However, administering a vasopressor can be detrimental when the heart is starting up by itself. The present embodiment may be advantageous in that it may enable an automated solution for providing decision support in terms of advising a caregiver in administering a vasopressor agent. The vasopressor agent signal may be based on combining an outcome from the one or more processes, so as to enable providing an advice on administration of a vasopressor agent. The combining may be similar to the combining of outcome from the one or more processes for providing the total probability of Return of Spontaneous Circulation described elsewhere in the present application.

In an embodiment, there is presented a device wherein the processor is arranged for selecting the one or more processes to be carried out within a plurality of one or more processes, such as wherein the plurality of one or more processes comprise one or more of processes A-D. By ‘selecting’ may be understood, that the processor has access to a plurality of processes, and is arranged for selecting which processes to carry out and which processes not to carry out, such as the selection depending on the circumstances, such as the receipt of additional data, such as defibrillation data. It may be understood that some processes are more suitable in one set of circumstances, while other processes are more suitable in other circumstances, such that no one single process is capable of yielding the best result in all circumstances. Therefore, it may be seen as an advantage, that the processor is capable of selecting the one or more processes, since it enables selecting the optimal processes for a given set of circumstances, thereby enabling providing an improved result.

It may be noted, that by selecting one or more of the processes, one basically describes an adaptive formula, e.g., Ptot=f(PA, PB, PC, PD), e.g., depending on additional data, such as period elapsed since defibrillation (the defib-timing t-t_defib) and/or the defibrillation number and/or the CPR data. One can capture such an adaptive formula in the form of

Ptot=f(PA,PB,PC,PD,t-t_defib,Compression Depth,Compression Force)

as earlier described. For example, a good selection could be to select processes B and C shortly after defibrillation (these respond quickest in approximately a minute), and process A after approximately 30 seconds and later. In another example: If one uses a memory element (finite state machine) one can keep track of the latest detected pulse rate. Whenever the pulse rate tends to merge with the compression frequency (using CPR data) the combiner can assign a lower importance to process A as this process is unreliable whenever the pulse rate and compression frequency coincide.

In an embodiment, there is presented a device wherein the input is furthermore arranged for receiving additional data representative of any one of:

CPR data, such as data indicative of timing of compressions, compression depth, compression velocity, compression acceleration, and/or compression force

defibrillation data, such as data indicative of timing of defibrillation, and/or

transthoracic impedance data,

and wherein the processor is arranged for accessing said additional data. Receipt of additional data may be beneficial in that it enables the processor to select which processes to carry out, and or enables that calculations carried out by the processor may take into account relevant additional data.

In a further embodiment, there is presented a device wherein the

calculation of the one or more parameters (PA, PB, PC, PD) indicative of a probability of Return of Spontaneous Circulation corresponding to an outcome of each process within the one or more processes,

and/or wherein the

calculation of the total probability (Ptot) of Return of Spontaneous Circulation based on the one or more parameters (PA, PB, PC, PD) indicative of a probability of Return of Spontaneous Circulation corresponding to an outcome of each process within the one or more processes,

is based at least partially on said additional data.

Basing said calculations on the additional data may be advantageous, in that it enables that said calculations may be optimized in dependence of the additional data.

In another further embodiment, there is presented a device wherein the selection of the one or more processes to be carried out within a plurality of one or more processes, such as wherein the plurality of one or more processes comprise one or more of processes A-D, is based at least partially on said additional data. It may be an advantage that the selection is based on additional data, since for example each of processes A-D are particularly suitable for given situations (or ‘circumstances’ or ‘challenges’), cf, the table inserted below, which elucidates the strengths of the processes, and thus highlights the synergy in combinations of them. A plus sign indicate that a process is suitable in overcoming a given challenge, two plus signs indicate that a process is particularly suitable in overcoming a given challenge, and a minus sign indicate that the strategy may be relatively less suitable in overcoming the corresponding challenge:

TABLE I

| | Challenge 1 | | | |
|---|---|---|---|---|
| | (indistin- | Challenge 2 | Challenge 3 | Challenge 4 |
| | guishable | (assess clin- | (irregular | (coinciding |
| | compressions | ical signif- | beating of | frequencies |
| | and heart in | icance of | the heart | of compres- |
| | time repre- | strength of | in start- | sions and |
| | sentation) | pulse) | up phase) | heart rate) |
| Process A | + + | + | − | − − |
| Process B | − | + | + + | − − |
| Process C | − | + | + | ++ |
| Process D | + | − | + | + |

In another further embodiment, there is presented a device wherein the selection of the one or more processes to be carried out within a plurality of one or more processes, such as wherein the plurality of one or more processes comprise one or more of processes A-D, is based at least partially on said additional data and TABLE I.

In another further embodiment, there is presented a device, wherein the plurality of one or more processes comprise

process A

### OR

process A and process C,

wherein in process A the scoring of each peak furthermore depends on:

the amplitude of the peak, such as where a higher score is given for a higher amplitude, and/or

the amplitude of the remaining peaks, such as where a higher score is given for a higher amplitude, which correspond to a harmonic of the peak or correspond to a sum or difference frequency between

1. the peak or harmonics of the peak and

2. a chest compression frequency or harmonics of the chest compression frequency. Advantages of each of these embodiments is given in the “exemplary embodiment relating to processes A and C” inserted in the end of the description. It may be understood in relation to this embodiment and/or process A in general, that obtaining a spectrally resolved representation of the photoplethysmograpy data may comprise employing an autoregressive (AR) model. It may be understood in relation to this embodiment and/or process A in general, that ‘photoplethysmography data’ may refer to raw photoplethysmography data or ‘photoplethysmography data which have been processed’, such as ‘photoplethysmography data wherein a compression component has been removed’, such as removed by subtracting an estimate of the compression component, wherein the estimate of the compression component may optionally be modelled by a harmonic series. It may be understood in relation to this embodiment and/or process A in general, that process A may comprise removal of a compression component from the photoplethysmography data, such as removal of the compression component by subtraction of an estimate of the compression component, wherein the estimate of the compression component may optionally be modelled by a harmonic series.

In a second aspect, the invention provides a system comprising a device according to the first aspect, wherein the system furthermore comprises one or more of:

an automated CPR device, such as an automated CPR device arranged for sending CPR data to the input of the device and wherein the processor is arranged for accessing said CPR data,

a defibrillator, such as a defibrillator arranged for sending defibrillator data and/or transthoracic impedance data and/or CPR data to the input of the device and wherein the processor is arranged for accessing said defibrillator data and/or said transthoracic impedance data and/or CPR data,

a memory unit arranged for storing data, such as adaptive data, arranged for modifying the calculation of the one or more parameters (PA, PB, PC, PD) indicative of a probability of Return of Spontaneous Circulation corresponding to an outcome of each process within the one or more processes.

An advantage of providing an automated CPR device may be that it enables CPR and/or that it enables obtaining CPR data. An advantage of providing a defibrillator may be that it enables defibrillation and/or that it enables obtaining defibrillator data. It may be understood, that defibrillator data may comprise CPR data, since a defibrillator often also records CPR data (like a compression force, acceleration, velocity and depth curve), which can also be sent to the processor. An advantage of providing a memory unit may be that it enables storage of CPR data and/or defibrillator data which may be used to modify calculations, such as parameters used in calculations, such as adaptive parameters used in calculations which can be adapted so as to modify (and improve) the calculations, such as the calculations of the one or more parameters.

In the present application, by ‘CPR data’ is understood any data providing information on the CPR procedure and/or CPR quality, such as timing of a compression, compression force, compression depth, compression velocity, compression acceleration, compression phase of a periodical compression sequence and/or compression frequency.

In an embodiment, there is presented a system comprising a device according to the first aspect, wherein the system is furthermore comprising a measurement unit for obtaining the photoplethysmograpy data from an associated patient, such as the measurement unit being a pulse oximeter. The measurement unit may be, e.g., a data storage device used for storing and retrieving digital information, such as a hard disk drive.

In a further embodiment, there is presented a system wherein the measurement unit is a pulse oximeter, such as a pulse oximeter comprising:

a light source for transmitting light of a first wavelength into an associated patient over a period of time,

a light detector that receives light of a first wavelength transmitted into the patient over a period of time,

a light source for transmitting light of a second wavelength into an associated patient over a period of time,

a light detector that receives light of a second wavelength transmitted into the patient over a period of time,

and wherein the pulse oximeter is arranged for generating a set of photoplethysmography data in response to the received light, and furthermore capable of sending the set of photoplethysmography data to the input of the device. Pulse oximeter is understood as is known in the art. A pulse oximeter may be understood to use at least two wavelengths, such as two wavelengths, such as a first wavelength at 660 nm, such as a second wavelength at 900 nm.

In an embodiment, there is presented a system comprising a communication unit for presenting signals from the output unit to a user, such as the Return of Spontaneous Circulation probability signal and/or the vasopressor agent signal and/or the measured pulse rate and/or the variability of said pulse rate. It may be understood that each of said signals may be presented in an effectively continuous or discretized manner. In a further embodiment, there is presented a system, wherein the communication unit comprises:

a display for visual communication, such as a computer screen, and/or

a loudspeaker for audio communication.

In a third aspect, the invention provides a method for determining a total probability (Ptot) of Return of Spontaneous Circulation during an associated CPR procedure which being performed on an associated patient, the method comprising:

obtaining a set of photoplethysmograpy data having been obtained from the associated patient during the CPR procedure,

carrying out one or more processes according to one or more predetermined algorithms, such as one or more automatable algorithms, such as one or more automatable processes which do not require user input, based on the photoplethysmography data, so as to determine one or more parameters (PA, PB, PC, PD) indicative of a probability of Return of Spontaneous Circulation corresponding to an outcome of each process within the one or more processes

providing the total probability (Ptot) of Return of Spontaneous Circulation based on the one or more parameters (PA, PB, PC, PD) indicative of a probability of Return of Spontaneous Circulation corresponding to an outcome of each process within the one or more processes,

providing a Return of Spontaneous Circulation probability signal based on the total probability (Ptot) of Return of Spontaneous Circulation.

It is noted that no steps of the method requires interaction with a patient's body and/or involvement of a medical practitioner.

In an embodiment, there is presented a method wherein the one or more processes comprise 1 or 2 or 3 or 4 of the processes within processes A-D.

In a fourth aspect, the invention provides a computer program, such as a computer program product, enabling a processor to carry out the method according to the third aspect. A computer program may be stored/distributed on a suitable medium, such as an optical storage medium or a solid-state medium supplied together with or as part of other hardware, but may also be distributed in other forms, such as via the Internet or other wired or wireless telecommunication systems.

It is appreciated that the same advantages and embodiments of the first aspect apply as well for the second aspect. In general the first and second aspects may be combined and coupled in any way possible within the scope of the invention. These and other aspects, features and/or advantages of the invention will be apparent from and elucidated with reference to the embodiments described hereinafter.

### BRIEF DESCRIPTION OF THE DRAWINGS

Embodiments of the invention will be described, by way of example only, with reference to the drawings, in which

FIG. 1 illustrates an embodiment with a system 110 comprising a device 100 (a ‘patient monitor’),

FIG. 2 illustrates two examples of the display of the monitor for a caregiver,

FIG. 3 illustrates a schematic flowchart according to an embodiment,

FIGS. 4-8 serve to support exemplary embodiments of process A-D and the calculation of the total probability of ROSC,

FIG. 9 shows a flowchart of an embodiment 964 of Process C,

FIG. 10 shows entropy of the infrared spectrum (0-400 BPM),

FIG. 11 shows infrared PPG DC as the dotted line,

FIG. 12 shows correlation between red (R) and infrared (IR),

FIG. 13 shows overview of a PPG based algorithm according to exemplary embodiment I,

FIG. 14 shows a flow chart of an iterative algorithm according to exemplary embodiment I,

FIG. 15 shows detection of individual chest compressions in a thranstoraic impedance (TTI) signal according to exemplary embodiment I,

FIG. 16 shows removal of the compression component from the PPG signal according to exemplary embodiment I,

FIG. 17 shows effective removal of compression components at the compression rate and its harmonics in the PPG signal according to exemplary embodiment I,

FIG. 18 shows a mean of the prediction error power relative to the compression-free PPG signal power as a function of AR model order according to exemplary embodiment I,

FIG. 19 shows data from a PR selection algorithm according to exemplary embodiment I,

FIG. 20 shows detection of baseline decrease according to exemplary embodiment I,

FIG. 21 shows detection of signs of a spontaneous pulse via the PPG signal during CPR after a successful defibrillation shock according to exemplary embodiment I.

### DESCRIPTION OF EMBODIMENTS

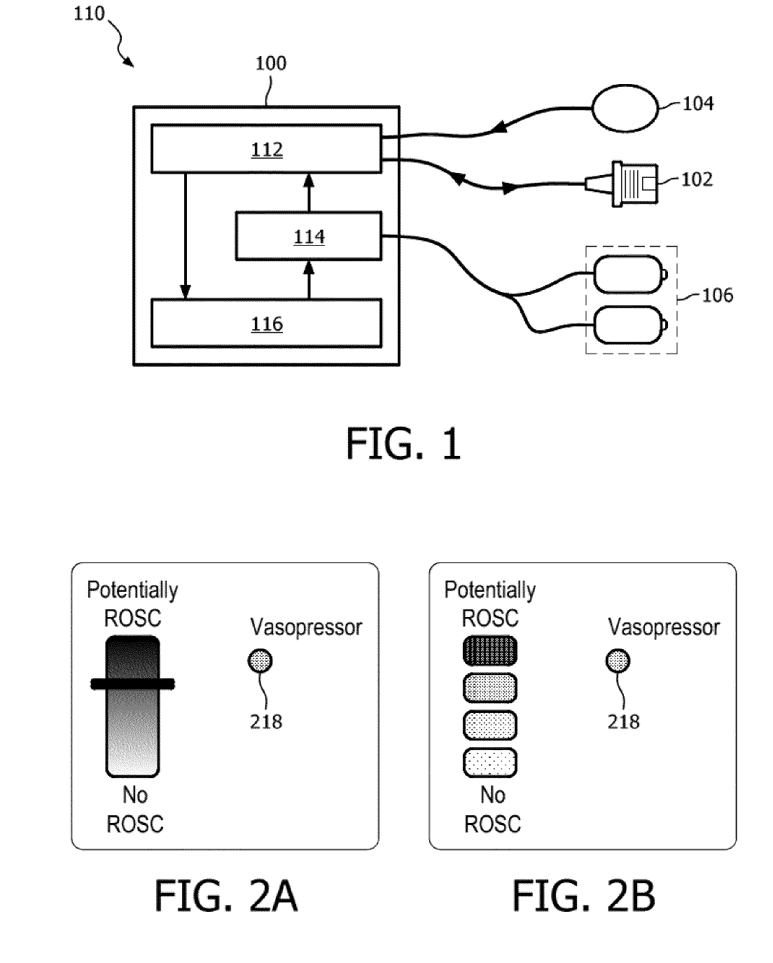

FIG. 1 illustrates an embodiment with a system 110 comprising a device 100 (a ‘patient monitor’), which in the present embodiment is also a defibrillation device in the sense that it comprises electronics 114 for controlling defibrillator pads 106, connected to a commercial PPG sensor 102 or pulse oximeter. The device contains a processor, such as electronic circuitry 112 with access to or comprising the one or more predetermined algorithms. The system also comprises a display 116. The device is connected or integrated with a defibrillator, such as a set of defibrillator pads 106. This allows the algorithm to know when the shock is given and to obtain information on the chest compressions via, e.g., a transthoracic impedance measurement. In that way, the algorithm may carry out an automated pre-shock calibration. The device may also be connected to an automated CPR device. The automated CPR device provides information to the algorithm on compression frequency, phase, and acceleration, velocity and depth. The PPG 102 sensor is equipped with an accelerometer 104 (illustrated independently in the figure). The accelerometer 104 provides information to the algorithm on compression frequency and compression pauses. In alternative embodiments, the system does not comprise, e.g., the defibrillator pads 106 and/or the CPR device and/or the accelerometer 104.

FIG. 2 illustrates two examples of the display of the monitor for a caregiver. FIG. 2A shows a gradual, continuous scale to indicate the likelihood of ROSC between no-ROSC and potentially ROSC. It also contains an indicator 218, such as a light emitting diode, that can provide a negative advice for administering a vasopressor, such as epinephrine. FIG. 2B is similar, except for showing a gradual, discrete scale to indicate the likelihood of ROSC.

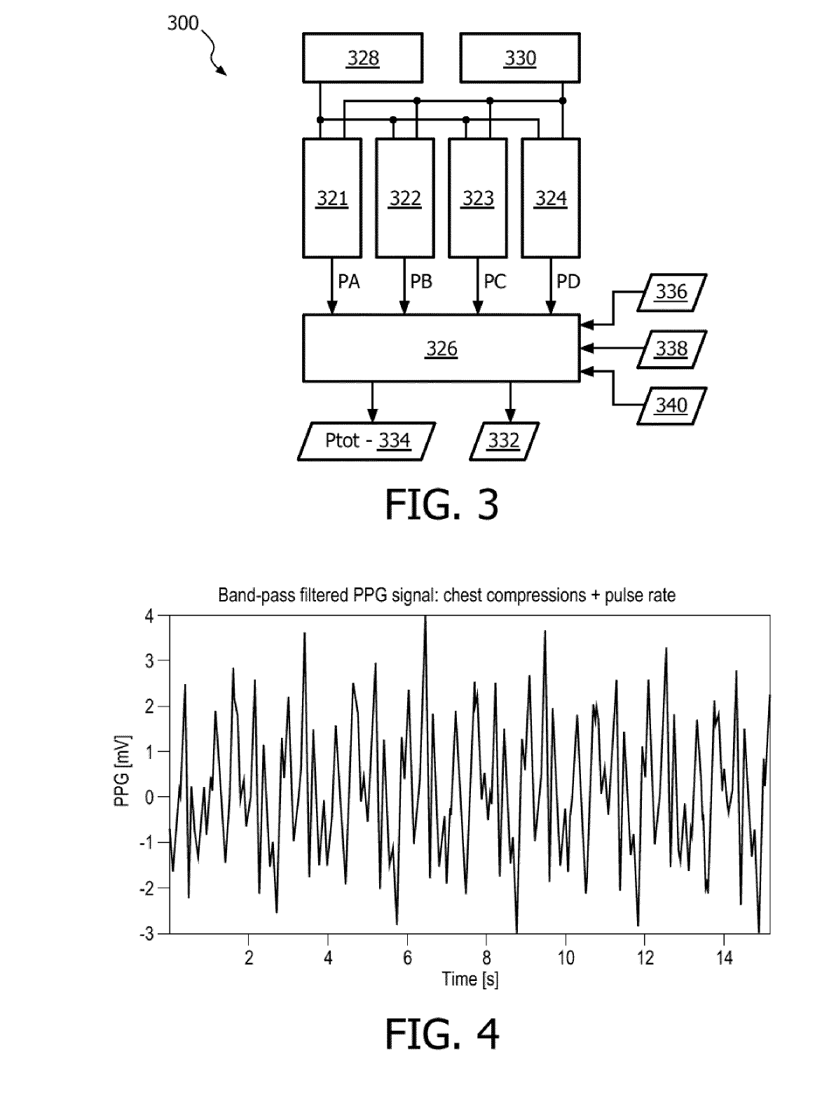

FIG. 3 illustrates a schematic flowchart according to an embodiment of a method 300 of the invention. It relies on four parallel PPG assessment strategies, such as embodiments of processes A-D: Advanced spectral peak identification 321, spectral entropy 322, PPG DC value 323, multi-wavelength correlations 324, corresponding to processes A-D, which each take as input a raw PPG signal at a primary wavelength 328 and a raw PPG signal at a secondary wavelength 330, and calculate respectively process parameters PA, PB, PC and PD that are then combined to compute the total probability of ROSC in parallel combiner 326 of all strategies, and is furthermore arranged to present advice 332 on administration of epinephrine or another vasopressor agent. Thus, the outcome of all individual, independent assessment strategies (i.e., each of the one or more processes) are combined, such as combined together with a confidence measure, to determine the total ROSC probability 334 as shown schematically in FIG. 3. It is also understood that the parallel combiner 326 of all strategies, may furthermore receive as input a defibrillator signal 336, CPR data 338, such as a signal from an automated CPR device, and an accelerometer signal 340.

FIGS. 4-8 serve to support exemplary embodiments of each of strategies 1-4/processes A-D, which are described in the following:

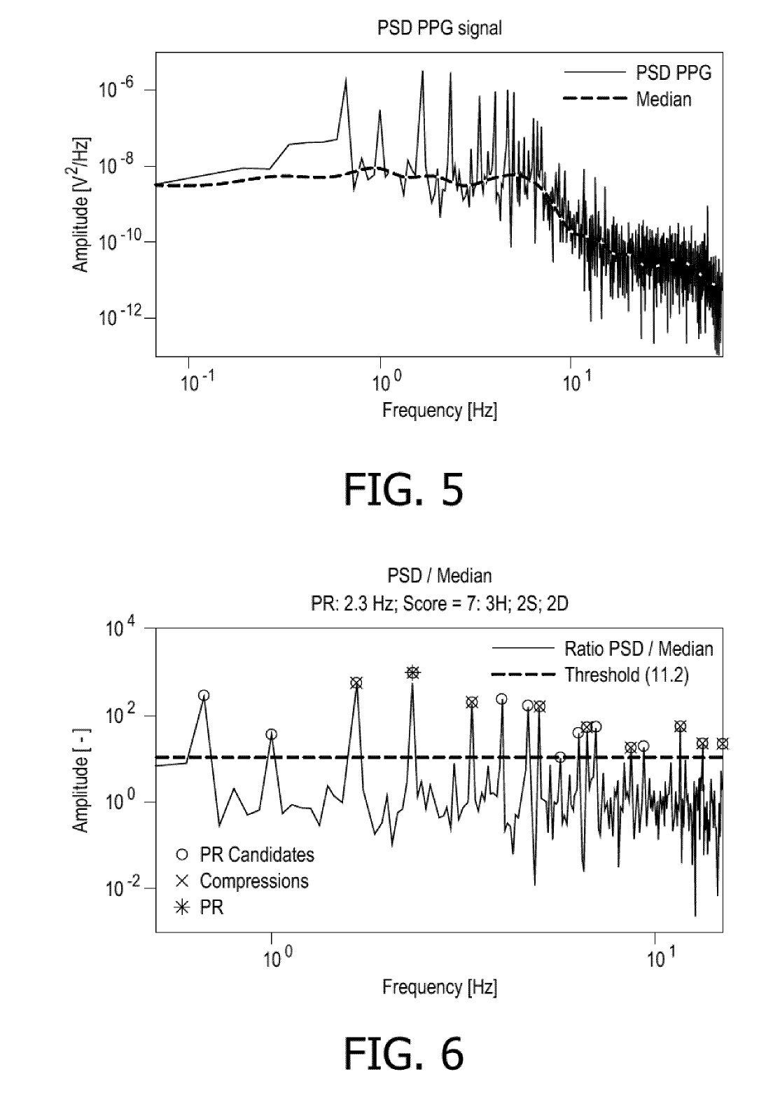

PPG-assessment according to an example according to Process A: Advanced spectral pulse analysis. The DC value of the PPG signal is removed first, as shown in FIG. 4. Next the power spectral density (PSD) of the PPG signal is determined (solid line in FIG. 5), and it is equalized by its baseline or minimum level (the dashed line in FIG. 5 shows the baseline, and the solid line in FIG. 6 shows the equalized spectrum). Subsequently, an adaptive thresholding technique is employed to determine the optimal threshold that separates weak and strong periodic components (dashed line in FIG. 6), to identify all strong periodic components (circles in FIG. 6). The chest compression frequency and its harmonics are recognized and not considered as possible pulse rate (PR) (crosses in FIG. 6). Chest compression frequencies are either known in case of automated CPR, or independently measured using, e.g., an accelerometer or transthoracic impedance. In the remaining set of peaks, referred to as PR candidates, the relationship between all candidates is determined via a scoring method. Each candidate receives a score equal to the number of harmonics and the number of interaction terms found in the set of candidates. Interaction terms are the sum and difference frequencies of the PR and the chest compression frequency and their harmonics, such as correspond to a sum or difference frequency between

1. the peak or harmonics of the peak and
2. a chest compression frequency or harmonics of the chest compression frequency.

These non-linear interaction terms have been observed in our measurement data and are now explicitly used to correctly identify the PR component in a set of PR candidates. As an example, in FIG. 6 the identified PR component (indicated by a star) has a score of seven, which results from three harmonics, two sum interaction terms and two difference interaction terms being present in the set of PR candidates. In another embodiment, before analyzing the spectral content of the PPG signal, the chest compression frequencies are removed from the PPG signal first, by e.g., making use of an accelerometer or transthoracic impedance measurement, or by e.g. using principal component analysis (PCA) or independent component analysis (ICA). In the Appendix a detailed description is provided of one embodiment of the advanced spectral pulse analysis. An overview of the figures is presented here:

FIG. 4 shows a band-pass filtered PPG signal during chest compressions when the mechanical activity of the heart has been restored. The data is thus understood to reflect both chest compressions and pulse rate.

FIG. 5 shows power spectral density (PSD) of the PPG signal shown in FIG. 4 (solid) and its baseline estimated via sliding-window median-filtering (dashed).

FIG. 6 shows the normalized PSD (solid), an optimal detection threshold (dashed) is used to detect strong periodic components (circles). In the resulting set of periodic components, frequencies related to chest compressions are directly recognized (crosses), and the remaining components are scored to identify the PR (pulse rate) component (star). (Note that the equalized spectrum is shown for a smaller frequency range than the frequency range of the PSD in FIG. 5).

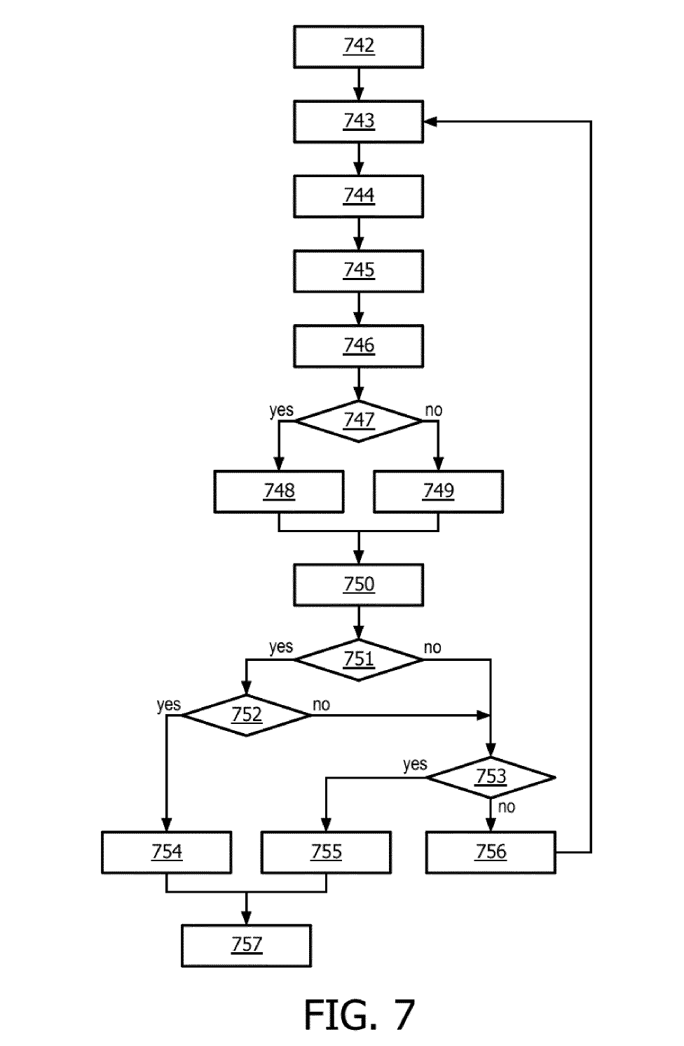

FIG. 7 shows a flowchart of an exemplary embodiment of process A, which may be referred to interchangeably as PPG-assessment strategy 1 which can be used when the compression frequency and its harmonics are first removed from the PPG signal, e.g., by adaptive filtering, that can make use of a reference signal, such as the transthoracic impedance. Furthermore, here it is assumed that the spectrum has been determined via autoregressive (AR) modeling, indicated as P_{AR}(f). The main idea of this algorithm is to score each peak in the spectrum based on the amplitude of the peak, and the amplitude of the peaks which are related harmonically or as an interaction term. The frequency for which this score is maximal is selected as PR. This effectively corresponds to an idea of scoring the peak by the amplitude, comprising adding to the amplitude of all peaks in the spectrum, the amplitude of the peaks which are related harmonically or as an interaction term, and selecting the frequency for which this summation is maximal as PR This algorithm has been based on the reference Hinich, M. J. (1982). Detecting a Hidden Periodic Signal When Its Period is Unknown, IEEE Transactions on Acoustics, Speech and Signal Processing, ASSP-30(5), 747-750, which is hereby included by reference in entirety. Furthermore, here the spectral peaks are taken into account in a recursive way, starting with the strongest peak, until based on the signal structure it has been decided which frequency component is the PR. The spectral peak selected by the algorithm in FIG. 7 is most likely the PR fundamental, because:

The strongest set of harmonics will be found for the PR fundamental, as it is the first component of the series.

If the pulse rate fundamental does not have any harmonics, it still can be recognized as the component right in the middle between the strongest interaction terms, e.g., f_PR+f_cmp and |f_PR−f_cmp|.

Here, the amplitude of the spectral components is relevant, to be able to recognize the strongest interaction terms. Furthermore, in this embodiment, a combination with spectral entropy (such as process B) and/or a change in PPG baseline (such as process C) and/or the amplitude of spectral peaks, such as the PR candidates, with respect to other spectral components may be preferred, to decide whether a spontaneous pulse is present in the PPG signal and whether the described recursive spectral peak analysis should be performed.

FIG. 7 more particularly describes:
742: Nₚₑₐₖₛ=2
743: Create set of frequencies {fₚ}:
   frequencies of Nₚₑₐₖₛ strongest peaks in P_{A} (f)
744: Create set of candidate PR frequencies {f_{cnd}}:
   frequencies in {fₙ} between 30 BPM and 300 BPM
745: For all frequencies in {f_{cnd}}:
   create set of elated frequencies {fᵣₑₗ} by searching in set {fₚ} for:
      fₕᵣₘ=2*f_{cnd}
      fₛᵤₘ=f_{cnd}+100
      f_{diff}=|f_{cnd}−100|
         where it may be understood that the compression frequency for this particular example is given by f_cmp=100 BPM,
746: For all frequencies in {f_{cnd}} with related fₛᵤₘ and f_{diff}:
   if [P_{AR}(fₛᵤₘ) P_{AR}(f_{cnd})] & [P_{AR}(f_{diff}) P_{AR}(f_{cnd})]
      then remove fₛᵤₘ and f_{diff} from {fᵣₑₗ}
747: For all frequencies in {f_{cnd}}: have related components been found?
748: Score(f_{cnd})=sum(P_{AR}([f_{cnd}, {fᵣₑₗ}]))
749: Score(f_{cnd})=0
750: Select maximum score
751: Maximum>0?
752: Single maximum found?
753: Nₚₑₐₖₛ=Number of identified peaks?
754: PR=f_{cnd} with maximum score
755: PR not Identified
756: Nₚₑₐₖₛ=Nₚₑₐₖₛ+1
757: Stop

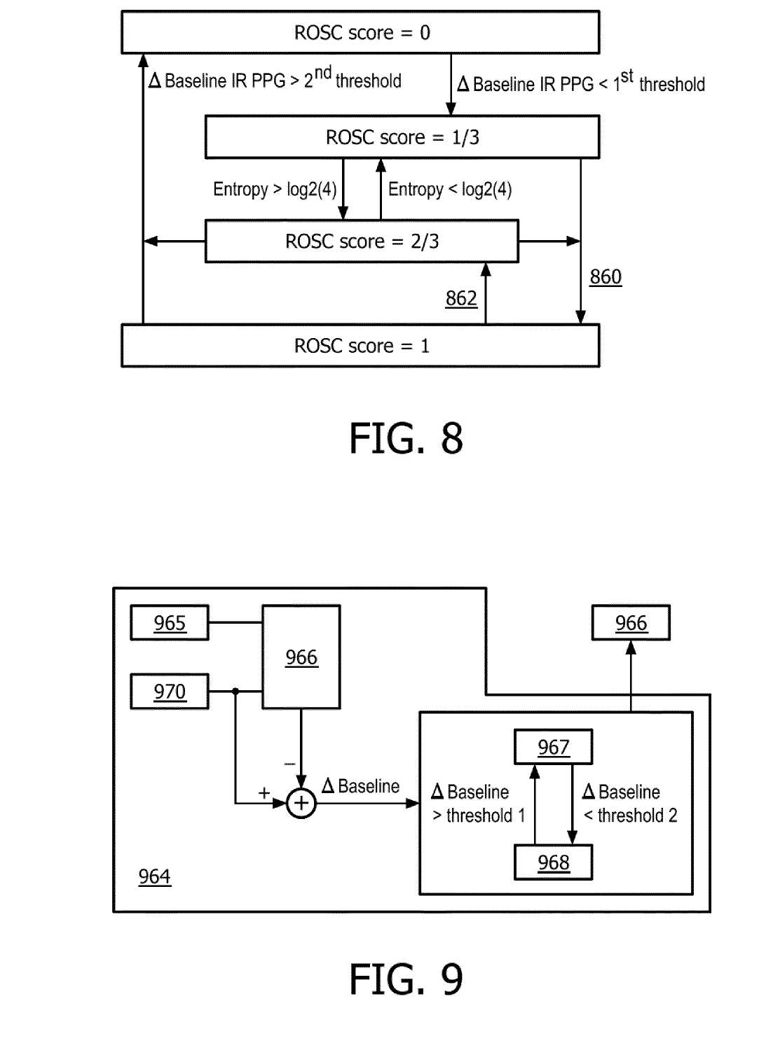

FIG. 8 shows a flowchart of a finite state machine representation of an embodiment on combining Process A, Process B, and Process C to compute a ROSC score, which may be seen as a number indicative of the probability of return of spontaneous circulation. The state of the finite state machine is memorized by keeping track of in which of the boxes with text “ROSC score=” the machine resides. The state of the finite state machine starts in box with ROSC-score is 0. Delta (Δ) Baseline Infrared (IR) represents the time derivative of the baseline PPG signal during compressions (possibly using CPR data to determine the periods in which compressions are present) averaged over 20 seconds. Motion of the sensor is easily detected by exceptionally large and abrupt changes in baseline in which case the baseline signal will be discarded. If the “Delta Baseline Infrared” crosses certain thresholds, such as determined in process C, the finite state machine can go into other states, for example ROSC score=⅓. Similarly, if the spectral entropy, computed in a Process B, crosses certain thresholds, the system can go into other states too, e.g., ROSC score=⅔. Similarly, the outcome of Process A on spectral peak identification can bring the system in the state with ROSC score=1, such as ‘Pulse rate detected’ 860 and ‘Pulse rate not detected’ 862 may each also affect the state/score.

FIG. 9 shows a flowchart of an embodiment 964 of Process C which makes use of memory units 966, 967, 968 and CPR data and defibrillation data, which may be received from an automated CPR device and a defibrillator with defibrillator pads as indicated by box 965. The process memorizes with primary memory unit 966 the PPG baseline at the point in time, such as at the moment or a finite period (such as 10 seconds), just before the defibrillation shock as a reference. The PPG baseline may be obtained with means 970 for obtaining PPG data, such as a pulse oximeter. Whenever the baseline of the PPG signal crosses (potentially different) thresholds for up and down (i.e., the change in baseline level, delta (Δ) baseline exceeds certain levels, such as Threshold 1 or Threshold 2), Process C 964 will give either 1 (as indicated by assigning 1 to process C parameter PC in the secondary memory unit 967) or 0 (as indicated by assigning 1 to process C parameter PC in the tertiary memory unit 968) as the number for process C parameter PC. This number can then later be combined with other Processes to compute a number indicative of the total (Ptot) probability of return of spontaneous circulation. It is understood, that the memory units 966, 967, 968, while shown separated for clarity, may be embodied as a single memory unit.

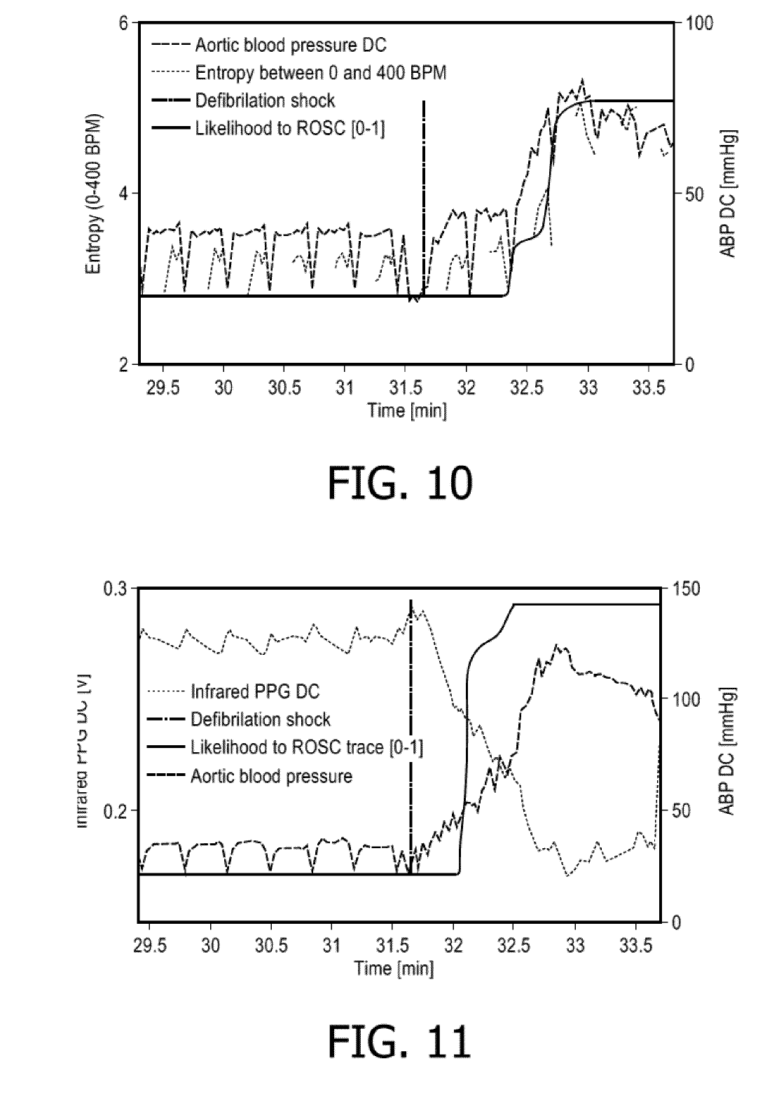

FIG. 10 shows entropy of the infrared spectrum (0-400 BPM), and in particular shows entropy between 0 and 400 BPM as the dotted line.

FIG. 11 shows infrared PPG DC as the dotted line.

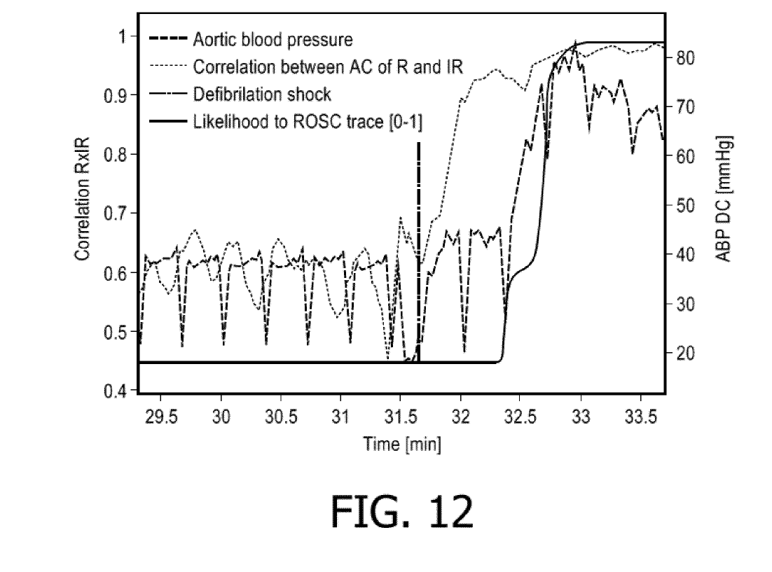

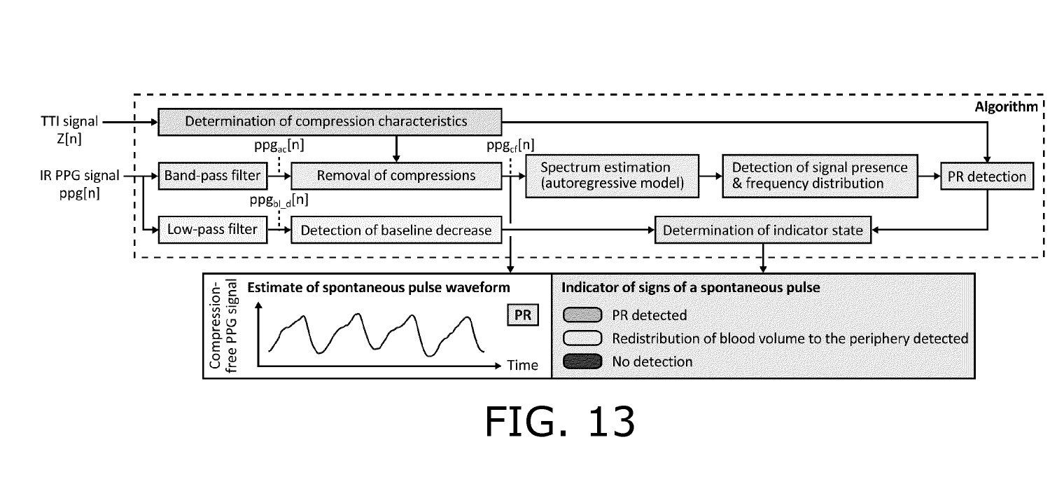

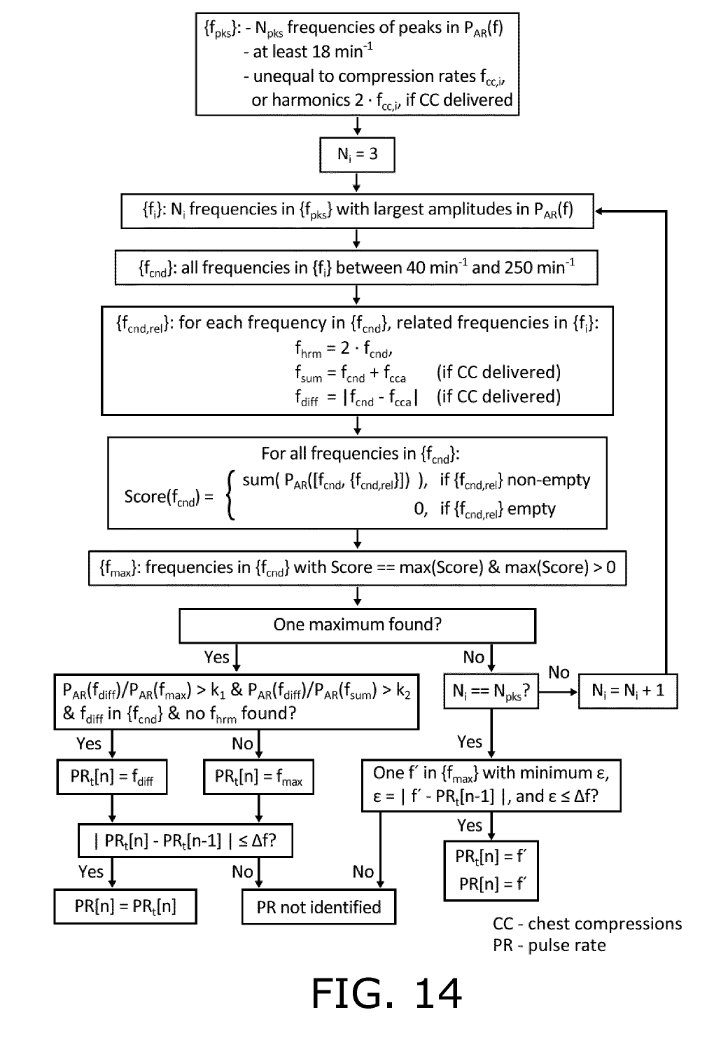

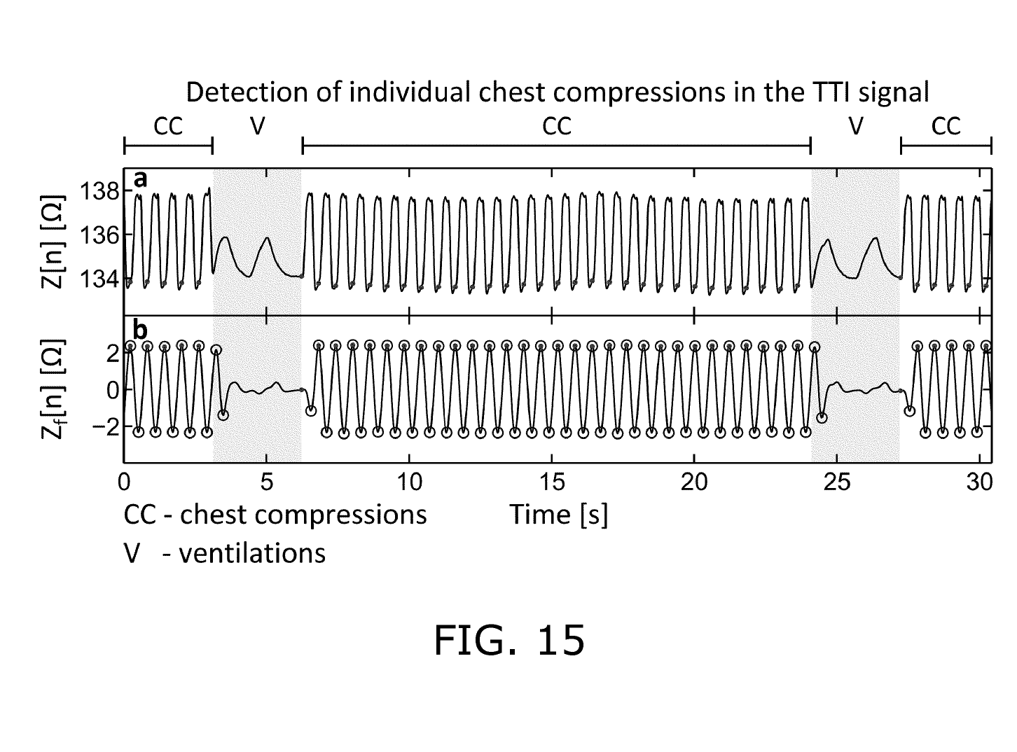

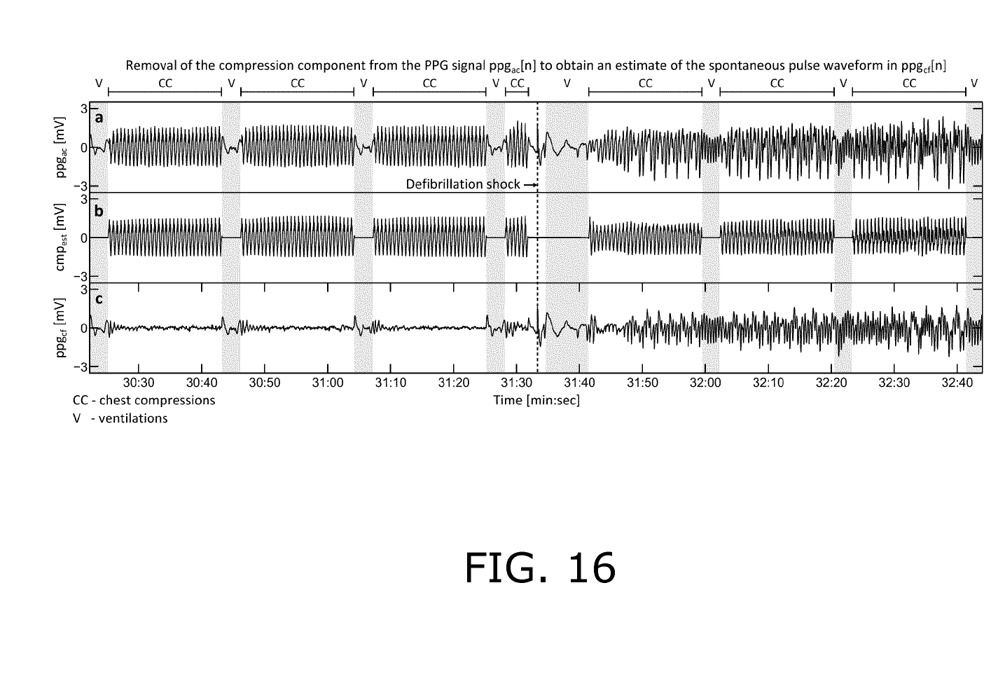

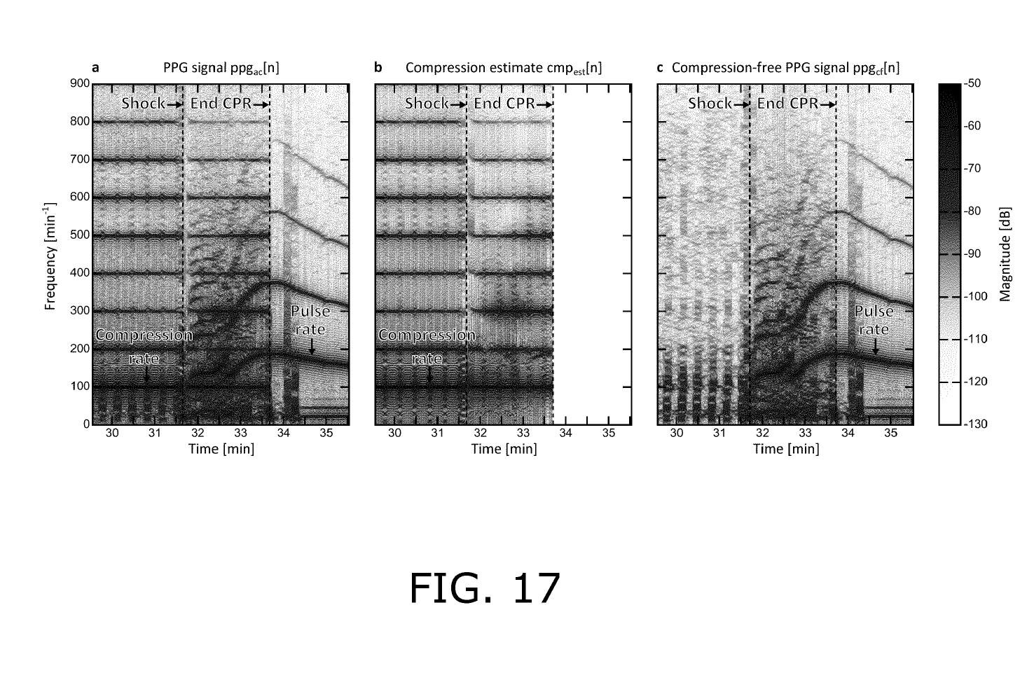

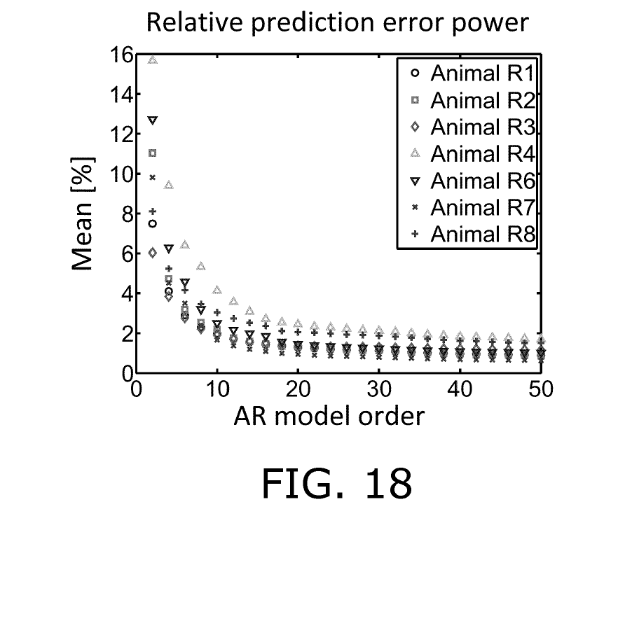

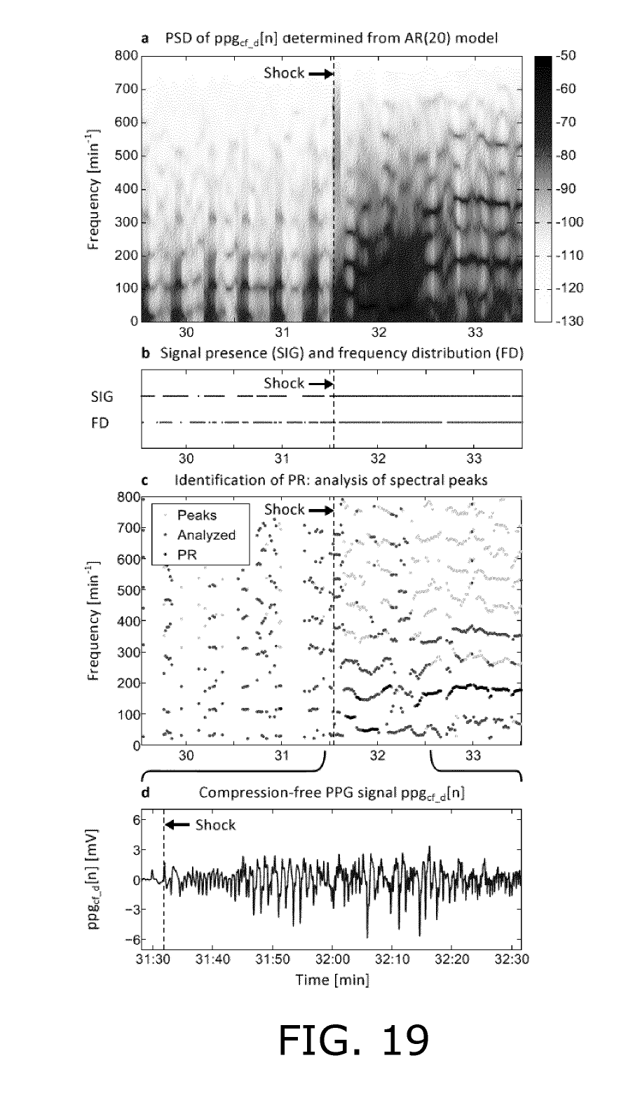

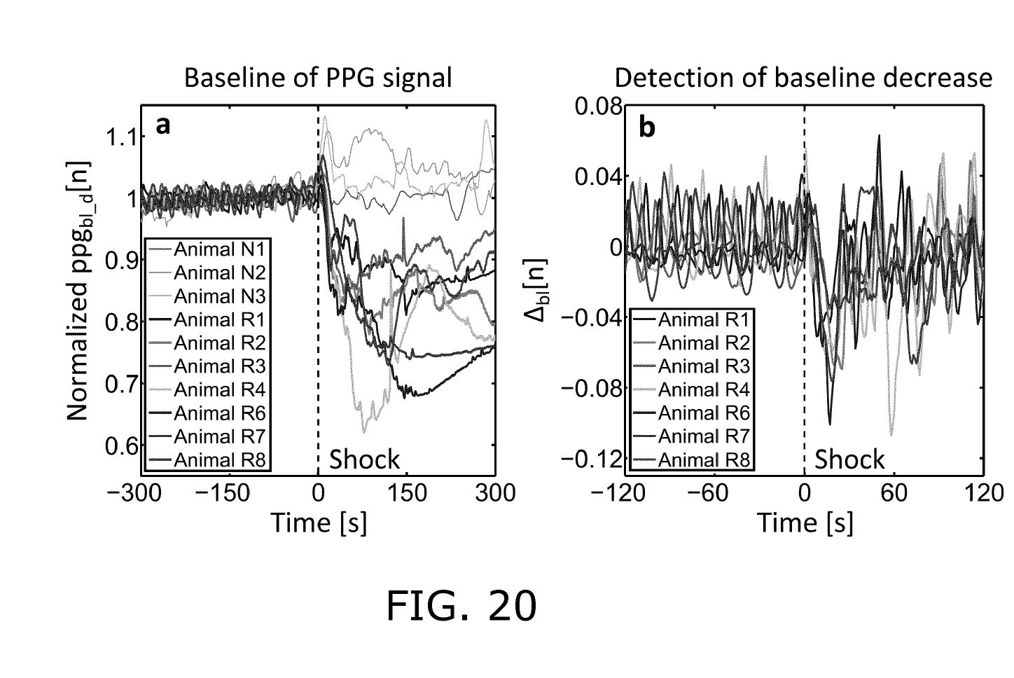

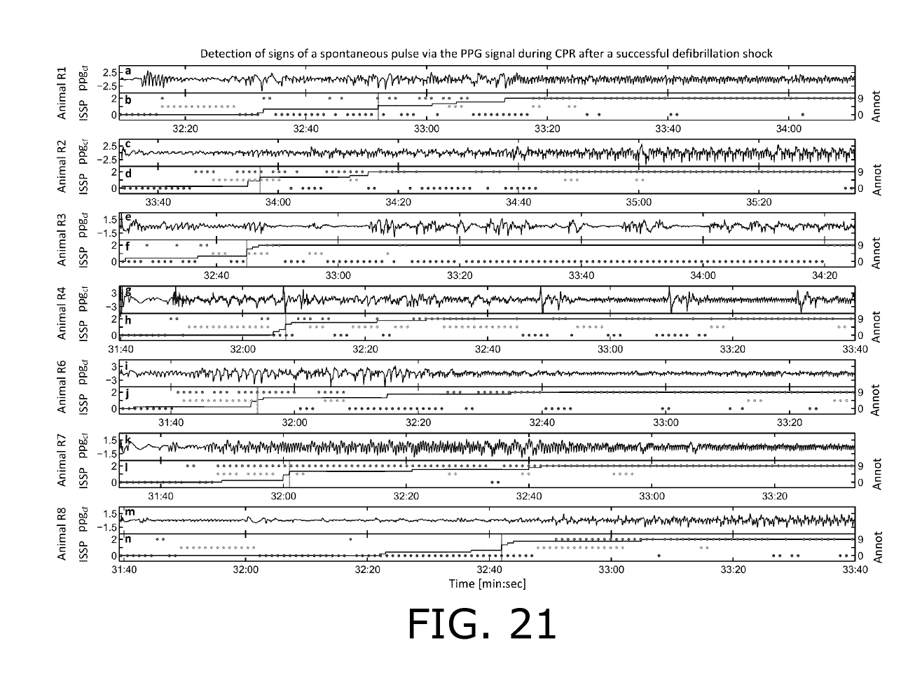

FIG. 12 shows correlation between red (R) and infrared (IR), and in particular shows correlation between the AC portion of the R and IR signals as the dotted line.

Note that FIGS. 10-12 each features a full-drawn, black (dark) curve representative of likelihood to ROSC (0-1). This curve is provided by interviewing nine expert physicians at the operating room, the emergency department, and the intensive care unit. The physicians were shown the electrocardiogram, the end-tidal CO₂ curve, the carotid artery flow and the arterial blood pressure (ABP) waveforms. The likelihood curve is a smoothed and normalized version of the number of physicians that indicated ROSC based on the above-mentioned curves that were presented to them. Furthermore, each of FIGS. 10-12 feature a “defibrillation shock” as indicated by a thick, vertical line, approximately at 31.7 min. Furthermore, FIG. 10 features “Aortic Blood Pressure DC” as the dashed line. Furthermore, each of FIGS. 11-12 feature “Aortic Blood Pressure” as the dashed line.

PPG-assessment according to an example according to Process B: Spectral entropy. As soon as the heart begins to start up, the complexity of the spectrogram quickly rises. This complexity is represented by the spectral entropy. This method is particularly sensitive in the transition phase from non-ROSC to ROSC. It is also sensitive to relatively weak pulses and irregular pulses. The performance of the strategy is best if the frequencies of the heart beat and the compressions do not coincide, since coinciding frequencies could hamper the quality of the outcome of the strategy. The strategy, works better in the post-transition phase (example of performance in FIG. 10 which shows an example of performance of the entropy strategy (dotted curve)). To improve the sensitivity for the start-up phase of the heart, it is recommended to determine the PPG spectrum from a time window resulting in a spectral resolution that accommodates the compression frequency, i.e., the time window should be chosen such that the compression frequency and its harmonics are integer multiples of the spectral resolution. This ensures that the energy of the compression sequence is confined to a limited number of bins in the spectrum, resulting in a low entropy when the PPG signal contains only compressions, and a distinct increase in entropy upon irregular activity in the PPG signal of which the energy spreads in the spectrum. Therefore, zero-padding should preferably not be applied either. In another embodiment, the compression frequencies are removed first, by, e.g., making use of an accelerometer or transthoracic impedance measurement, or by, e.g., using principal or independent component analysis, leading to nearly maximum entropy when no spontaneous pulse is present, and to a significant and sustained decrease in entropy when a spontaneous pulse has developed. In this second embodiment the time window from which the spectrum is determined is less relevant.

PPG-assessment according to an example according to Process C: PPG DC value. Changes in the PPG DC value reflect changes in the mean blood volume and/or the venous oxygen saturation. At full ROSC, the blood pressure has been restored and blood volumes have been redistributed, which results in larger blood volume at the sensor site corresponding to a lower DC value of, e.g., the infrared PPG signal. Furthermore, at full ROSC, restoration of tissue perfusion causes the venous oxygen saturation to increase back to normal level, resulting in an increase in the DC level of, e.g., the red PPG signal. This method does not depend on heart beats and is not compromised if the heart beat and the compression frequency coincide (example of performance in FIG. 11 which shows an example of performance of the PPG-DC strategy (dotted curve negatively correlates with the likelihood of ROSC)).

PPG-assessment according to an example according to Process D: Multi-wavelength correlations. Multi-wavelength correlations were discovered to reflect the level of peripheral perfusion and venous oxygen saturation. If blood pressure is low (before ROSC), the micro-vascular perfusion at the skin surface is low and the venous oxygen saturation is low due to an insufficient supply of oxygen, which results in an apparent shift (delayed) of the “red” PPG signal (660 nm) with respect to the “infrared” PPG signal (890 nm). As soon as the blood micro perfusion increases after ROSC, the red and infrared PPG signals become highly correlated. This method may thus even utilize the shape of the compression artefacts in the PPG signals (example of performance in FIG. 12 which shows an example of performance of the strategy of the multi-wavelength correlations (dotted curve)).

In the following, a more detailed description of one embodiment of the advanced spectral pulse analysis, corresponding to an embodiment of process A, is presented.

The advanced spectral pulse analysis detects periodic components in the PPG spectrum via an adaptive thresholding technique, and subsequently identifies the pulse rate (PR) component amongst the detected periodic components by analyzing the relationship between the detected periodic components. The advanced spectral analysis comprises the steps:

1. Band-pass filtering is applied to the PPG signal first to remove the baseline and higher-frequency components. The PPG signal's baseline can strongly fluctuate due to large variations in tissue blood volume, and can consequently mask periodic components in the spectrum. FIG. 4 shows a typical time trace of a band-pass filtered PPG signal during chest compressions, when the mechanical activity of the heart has been restored.
2. Subsequently, the spectrum of the PPG signal is determined and equalized to facilitate detecting the periodic components. Equalization of the spectrum can for instance be done by normalizing the spectrum by its baseline, which can be determined by applying a sliding-window median-filter to the spectrum. A convenient window-length of the median-filter can for instance be the chest compression frequency. FIG. 5 shows the spectrum of the band-pass filtered PPG signal of FIG. 4 (solid line), and its baseline as obtained by median-filtering (dashed line).
3. Periodic components are then detected in the equalized PPG spectrum by selecting all frequency components larger than a threshold, which is adapted over time to each specific spectrum. The detection threshold is for instance optimal with respect to an optimization criterion which tries to identify two classes with minimum intra-class variance and maximum inter-class variance (e.g., cf., the method described in the reference “Otsu, N. A Threshold Selection Method from Gray-Level Histograms. IEEE Transactions on Systems, Man, and Cybernetics, SMC-9(1), 62-66 (1979)” which reference is hereby incorporated by reference in its entirety). The optimization criterion is applied to a frequency range of interest (e.g., 0.5 Hz-15 Hz) and to an amplitude range of interest (e.g., larger than one). Furthermore, the amplitude range is converted to a logarithmic scale first, to prevent too much influence from outliers. The optimal threshold thus determined separates the strong periodic components in the magnitude frequency spectrum from all weaker components. FIG. 6 shows the equalized spectrum (solid), the optimal detection threshold (dashed), and all identified periodic components (circles). In an alternative implementation, via principal or independent component analysis and using accelerometer signals or a transthoracic impedance signal, the compression components can be identified in the PPG signal, and ignored in the subsequent analysis.
4. Spurious peaks which have been detected in the previous step can be partly removed via morphological operations applied to subsequent spectra. These methods can be used to remove spurious peaks caused by the windowing effect of the spectral analysis, and to remove peaks which are not persistent over time or have a too narrow spectral width to be a pulse rate component. The remaining periodic components identified are considered a set of PR (pulse rate) candidates.
5. The set of PR (pulse rate) candidates thus obtained is analyzed to identify the PR component. In case of automated CPR, the chest compression frequency and its harmonics are known and therefore can be directly recognized in the set of PR candidates. An additional accelerometer or a transthoracic impedance signal can be used as well to obtain information on the compression frequency and possible compression pauses. The accelerometer or transthoracic impedance signal can furthermore be used in combination with PCA or ICA to recognize the compression frequencies present in the PPG signal. All components in the set of candidates related to chest compressions are indicated by crosses in FIG. 6. Next, the relationship is analyzed between the remaining PR candidates. For each candidate, it is determined how many harmonics are present in the set, and how many interaction terms between the potential PR and chest compression frequencies can be found. Interaction terms are the sum and difference frequencies of the potential PR and the chest compression frequency and their harmonics. Each PR candidate receives a score equal to the number of relationships found in the set of candidates. The periodic component with the highest score is selected as PR. In FIG. 6 the PR component (indicated by a star) has a score of 7, which results from three harmonics, two sum interaction terms and two difference interaction terms being present in the set of PR candidates.
6. In case multiple PR candidates have the same maximum score, the PR can be identified by subsequently applying the following steps:

a. Try selecting the PR candidate that has both sum and difference interaction terms.

b. Otherwise try selecting the PR candidate in the preferred frequency range (e.g., 1 Hz-3 Hz).

c. Otherwise try selecting the PR candidate having the strongest signature in the spectrum, i.e., the PR candidate of which the sum of the (normalized) amplitudes of all associated spectral components is maximal.

d. Otherwise selecting the PR candidate having the lowest frequency.

7. Weak spontaneous pulses may not have harmonics or interaction terms in the PPG spectrum. These will have a score of zero, but can be detected if a score is assigned when the rate of such a weak pulse has been consistently detected in a number of subsequent spectra.

### Exemplary Embodiment I Relating to Processes A and C

The present example relates to an exemplary embodiment employing process A and process C. We defined a spontaneous pulse in the PPG signal as a (quasi-)periodic feature resulting from cardiac contractions. Here, a spontaneous pulse may be palpable or impalpable. The algorithm development has been based on pre-clinical data from [20]. Signs of a spontaneous pulse were detected using a compression-free PPG signal and the baseline of the PPG signal. The compression-free PPG signal, containing an estimate of the spontaneous pulse waveform, was obtained by removing the compression component, modeled by a harmonic series. The fundamental compression rate and phase of this series were derived from the transthoracic impedance (TTI) signal. The TTI signal had been measured between the defibrillation pads, as common in defibrillators. The PR was determined from the frequency spectrum of the compression-free PPG signal. Restoration of the heart beat could also be detected from a decrease in the baseline of the PPG signal, presumably caused by a redistribution of blood volume to the periphery. The algorithm indicated signs of a spontaneous pulse when a PR or a decrease in the baseline was detected. Note that the present example is self-contained in terms of literature references, and references to tables anf figures, where figures mentioned in the present example correspond to the figures in the list of figures having a figure number being 12 numbers higher.

To sum up, there is provided a device (100) and method for determining a total probability (Ptot) of Return of Spontaneous Circulation (ROSC) during an associated CPR procedure, which is being performed on an associated patient, comprising an input for receiving a set of photoplethysmograpy data (328, 330) having been obtained from the associated patient during the CPR procedure, and a processor (112) being arranged for carrying out one or more processes according to one or more predetermined algorithms (321, 322, 323, 324) so as to calculate the total probability (Ptot) of ROSC based on the one or more parameters, wherein the one or more processes are each, and/or in combination, being arranged for overcoming challenges derived from the CPR process, such as arbitrary signals not related to return of spontaneous circulation. In embodiments, the device and method relies on a plurality of processes in determining the total probability of ROSC.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. A single processor or other unit may fulfil the functions of several items recited in the claims. The mere fact that certain measures are recited in mutually different dependent claims does not indicate that a combination of these measured cannot be used to advantage. Any reference signs in the claims should not be construed as limiting the scope.

1. A device for determining a total probability of Return of Spontaneous Circulation during an associated CPR procedure which is being performed on an associated patient, the device comprising:
an input for receiving a set of photoplethysmograpy data having been obtained from the associated patient during the CPR procedure,
a processor being arranged for
accessing the photopletysmography data, wherein the processor is further arranged for
carrying out one or more processes A, B, C and/or D, wherein process A comprises:
i. obtaining a spectrally resolved representation of the photoplethysmograpy data,
ii. identifying peaks in the spectrally resolved representation,
iii. identifying a chest compression frequency,
iv. scoring each peak, where a higher score is given where a higher number of remaining peaks which correspond to a harmonic of the peak or correspond to a sum or difference frequency between
1. the peak or harmonics of the peak and
2. a chest compression frequency or harmonics of the chest compression frequency,
v. calculating a pulse rate within the data based on the peak with the highest score,
vi. determining a process A parameter (PA) indicative of a probability of Return of Spontaneous Circulation based on said pulse rate
process B comprises:
i. obtaining a spectrally resolved representation of the photoplethysmograpy data for determining a measure of order of the photoplethysmography data, and
ii. calculating a process B parameter (PB) indicative of a probability of Return of Spontaneous Circulation based on said measure of order,
process C comprises:
i. determining a low-frequency value of the photoplethysmography data, and
ii. calculating a process C parameter (PC) indicative of a probability (PC) of Return of Spontaneous Circulation based on said low-frequency value, and
process D comprises
i. enabling receipt of the set of photoplethysmograpy data, where the set of photoplethysmography data is a set of photoplethysmography data obtained at different wavelengths,
ii. determining a level of correlation between the set of photoplethysmography data obtained at different wavelengths, and
iii. calculating a process D parameter (PD) indicative of a probability (PD) of Return of Spontaneous Circulation based on said level of correlation
wherein the calculated parameters PA, PB, PC, PD are indicative of a probability of Return of Spontaneous Circulation corresponding to an outcome of each process within the one or more processes, and
calculating the total probability (Ptot) of Return of Spontaneous Circulation based on the one or more parameters (PA, PB, PC, PD) indicative of a probability of Return of Spontaneous Circulation corresponding to an outcome of each process within the one or more processes,
an output arranged for providing a Return of Spontaneous Circulation probability signal based on the total probability (Ptot) of Return of Spontaneous Circulation.

2. A device according to claim 1, wherein the processor is furthermore arranged for
calculating a risk parameter indicative of a risk that administration of a vasopressor agent would have negative effects, the risk parameter being based on the one or more parameters (PA, PB, PC, PD) indicative of a probability of Return of Spontaneous Circulation corresponding to an outcome of each process within the one or more processes,
and wherein the output is furthermore arranged for
providing a vasopressor agent signal based on the risk parameter.

3. A device according to claim 1, wherein the input is furthermore arranged for receiving additional data representative of any one of:
CPR data
defibrillation data and/or
transthoracic impedance data,
and wherein the processor is arranged for accessing said additional data and wherein the
calculation of the one or more parameters (PA, PB, PC, PD) indicative of a probability of Return of Spontaneous Circulation corresponding to an outcome of each process within the one or more processes,
and/or wherein the
calculation of the total probability (Ptot) of Return of Spontaneous Circulation based on the one or more parameters (PA, PB, PC, PD) indicative of a probability of Return of Spontaneous Circulation corresponding to an outcome of each process within the one or more processes,
is based at least partially on said additional data.

4. A device according to claim 1, wherein in process A the scoring of each peak furthermore depends on:
the amplitude of the peak, such as where a higher score is given for a higher amplitude, and/or
the amplitude of the remaining peaks, such as where a higher score is given for a higher amplitude, which correspond to a harmonic of the peak or correspond to a sum or difference frequency between
1. the peak or harmonics of the peak and
2. a chest compression frequency or harmonics of the chest compression frequency.

5. A system comprising a device according to claim 1, wherein the system furthermore comprises one or more of:
an automated CPR device, such as an automated CPR device arranged for sending CPR data to the input of the device and wherein the processor is arranged for accessing said CPR data,
a defibrillator, such as a defibrillator arranged for sending defibrillator data and/or transthoracic impedance data and/or CPR data to the input of the device and wherein the processor is arranged for accessing said defibrillator data and/or said transthoracic impedance data and/or CPR data,
a memory unit arranged for storing data arranged for modifying the calculation of the one or more parameters (PA, PB, PC, PD) indicative of a probability of Return of Spontaneous Circulation corresponding to an outcome of each process within the one or more processes.

6. A method for determining a total probability (Ptot) of Return of Spontaneous Circulation during an associated CPR procedure which being performed on an associated patient, the method comprising:
obtaining a set of photoplethysmograpy data having been obtained from the associated patient during the CPR procedure,
carrying out one or more processes A, B, C and/or D
wherein
process A comprises:
i. obtaining a spectrally resolved representation of the photoplethysmograpy data,
ii. identifying peaks in the spectrally resolved representation,
iii. identifying a chest compression frequency,
iv. scoring each peak, where a higher score is given where a higher number of remaining peaks which correspond to a harmonic of the peak or correspond to a sum or difference frequency between
1. the peak or harmonics of the peak and
2. a chest compression frequency or harmonics of the chest compression frequency,
v. calculating a pulse rate within the data based on the peak with the highest score,
vi. determining a process A parameter (PA) indicative of a probability of Return of Spontaneous Circulation based on said pulse rate
process B comprises:
i. obtaining a spectrally resolved representation of the photoplethysmograpy data for determining a measure of order of the photoplethysmography data, and
ii. calculating a process B parameter (PB) indicative of a probability of Return of Spontaneous Circulation based on said measure of order,
process C comprises:
i. determining a low-frequency value of the photoplethysmography data and
ii. calculating a process C parameter (PC) indicative of a probability (PC) of Return of Spontaneous Circulation based on said low-frequency value, and
process D comprises
i. enabling receipt of the set of photoplethysmograpy data, where the set of photoplethysmography data is a set of photoplethysmography data obtained at different wavelengths,
ii. determining a level of correlation between the set of photoplethysmography data obtained at different wavelengths, and
iii. calculating a process D parameter (PD) indicative of a probability (PD) of Return of Spontaneous Circulation based on said level of correlation
providing the total probability (Ptot) of Return of Spontaneous Circulation based on the one or more parameters (PA, PB, PC, PD) indicative of a probability of Return of Spontaneous Circulation corresponding to an outcome of each process within the one or more processes,
providing a Return of Spontaneous Circulation probability signal based on the total probability (Ptot) of Return of Spontaneous Circulation.

7. A computer program enabling a processor to carry out the method of claim 6.
